(19) United States
(12) Patent Application Publication
(10) Pub. No.: US 2011/0306065 A1
(43) Pub. Date: Dec. 15, 2011

(54) USE OF AN ANTIBODY AND A RARE-EARTH BASED CRYSTAL

(75) Inventors: Joseph Friedberg, Philadelphia, PA (US); Franz Fogt, Gladwyne, PA (US)

(73) Assignee: The Trustees of the University of Pennsylvania, Philadelphia, PA (US)

(21) Appl. No.: 13/129,090

(22) PCT Filed: Nov. 12, 2009
(86) PCT No.: PCT/US2009/064243

§ 371 Date: Aug. 30, 2011

Related U.S. Application Data
(60) Provisional application No. 61/114,040, filed on Nov. 12, 2008.

(51) Int. Cl. G01N 21/75; C12M 1/42; B82Y 15/00

(52) U.S. Cl. 435/7.25; 435/287.2; 977/700; 977/834; 977/902; 977/890

(57) ABSTRACT

The present invention provides compositions and methods for the detection of red blood cells antigens. The methods and compositions are based on antibodies, serving as primary antibodies or secondary antibodies, that are labeled with a rare-earth based crystal. Methods utilizing antibodies labeled with a rare-earth based crystal enhance the efficiency and accuracy of blood compatability tests and red blood cells phenotyping.

### CROSS REFERENCE TO RELATED APPLICATIONS

This application claims priority to U.S. Provisional Patent Application 61/114,040, filed Nov. 12, 2008, which is incorporated herein by reference in its entirety.

### FIELD OF THE INVENTION

This invention provides compositions and methods comprising an antibody labeled with a rare-earth based crystal.

### BACKGROUND OF THE INVENTION

More than 18.5 million units of red blood cells (RBCs) are collected in the United States each year, and must be antigen-typed prior to use in transfusion. Every unit is typed for ABO blood group and RH blood group. This is usually sufficient when patients receive blood transfusions. In a significant number of patients, however, there are antibodies present against specific blood sub groups (14-20 important subgroups) and the patient must receive blood which does not have that specific blood sub-group. Also, some patients may have to receive many blood transfusions throughout their life time and should receive blood with blood subgroups as closely to the patient's own as possible, in order to avoid to sensitize the patient against future blood transfusions. Very frequently, patients have a combination of unexpected antibodies and among the ABO typed blood donation bags, those without the specific blood groups against which the patient has antibodies, must be sought. If this is one antigen, the search may be short and only 3 to 6 units have to be tested. If the combination of antibodies is rarer, many donations have to be tested to find an antigen free unit. Often, this search can stretch nationwide. Each antigen typing is a hand process or a semiautomatic identification process which is extremely time consuming. Conventionally it involves mixing a small sample of RBCs with antibodies directed against RBC surface antigens. The presence of the surface antigens is inferred from the development of agglutination. Although this method is simple, labor-intensive test that limits the number of antigens for which RBCs can be typed. Typically RBCs are typed for two groups of antigens: ABO and Rh. Although these antigen groups are associated with the most severe forms of acute hemolytic transfusion reaction, there are hundreds of blood group antigens, at least 30 of which can induce a clinically-significant immune reaction. Because donor and recipient RBCs are usually only matched for ABO and Rh(D) antigens, most people are transfused with blood that is incompatible for one or more other RBC antigen groups.

The receipt of RBCs that exhibit foreign antigens usually stimulates an immune response (alloimmunization) in the recipient. Alloantibodies speed the removal of transfused RBCs from the circulation, reducing the effectiveness of the transfusion, and increasing the risk of non-hemolytic febrile transfusion reactions and allergic reactions. Although patients may be screened for the presence of prior alloantibodies before they receive a transfusion, the presence of antibodies can be difficult to interpret, and low titers of alloantibodies may not be detected. A prospective strategy for ensuring better donor and recipient blood compatibility across a broader number of antigen groups is what is needed. The development of new antibody reagents may be the key to developing homogeneous, multiplexed assays that could be used for blood typing, and other research and diagnostic applications.

In certain diseases or conditions an individual's blood may contain IgG antibodies that can specifically bind to antigens on the red blood cell (RBC) surface membrane, and their circulating red blood cells (RBCs) can become coated with IgG alloantibodies and/or IgG autoantibodies. Complement proteins may subsequently bind to the bound antibodies. The direct Coombs test is used to detect these antibodies or complement proteins that are bound to the surface of red blood cells; a blood sample is taken and the RBCs are washed (removing the patient's own plasma) and then incubated with antihuman globulin (also known as “Coombs reagent”). If this produces agglutination of RBCs, the direct Coombs test is positive, a visual indication that antibodies (and/or complement proteins) are bound to the surface of red blood cells.

A crystal can be characterized by the symmetry operations that leave its structure invariant. These can include rotation about an axis through a specific angle, reflection through a plane, inversion through a point, translations by a unit cell dimension, and combinations of these. For a periodic structure, the only allowable rotational symmetries are 2-fold, 3-fold, 4-fold, and 6-fold. A quasicrystal is a solid which yields a sharp diffraction pattern but has rotational symmetries (such as 5-fold or 10-fold) which are inconsistent with a periodic arrangement of atoms

### SUMMARY OF THE INVENTION

In one embodiment, the present invention provides a rare-earth ion doped, upconverting nanocrystal, comprising: a. a host molecule; b. a rare earth ion sensitizer; c. a rare earth ion emitter; and d. a coordination ligand capping the rare-earth ion doped nanocrystal, wherein the coordination ligand forces a substantially pure phase on the nanocrystal.

In another embodiment, the present invention further provides a functionalized rare-earth ion doped, upconverting nanocrystal comprising: a host molecule; b. a rare earth ion sensitizer; c. a rare earth ion emitter; and d. a coordination ligand capping the rare-earth ion doped nanocrystal, wherein the coordination ligand forces a substantially pure phase on the nanocrystal; and e. a functionalizing coating, wherein the functionalizing coating does not affect the optical properties of the nanocrystal.

In another embodiment, the present invention further provides a method of phenotyping red blood cells, comprising the steps of: obtaining a blood sample; b. contacting the blood sample with a composition comprising a first functionalized nanocrystal operably linked to an antibody or a functional fragment thereof, wherein the functionalized nanocrystal comprises:. a host molecule; a rare earth ion sensitizer; c. a rare earth ion emitter; a coordination ligand capping the rare-earth ion doped nanocrystal, wherein the coordination ligand forces a substantially pure phase on the nanocrystal; and. a functionalizing coating, wherein the functionalizing coating does not affect the optical properties of the nanocrystal; exposing the blood sample contacted with the functionalized nanocrystal operably linked to an antibody or a functional fragment thereof to an electromagnetic radiation source; and detecting an optical emission frequency, wherein the antibody or fragment thereof is specific against a red blood cells antigen, thereby phenotyping red blood cells.

In another embodiment, the present invention further provides a method of matching donated red blood cells to a recipient comprising the steps of: Obtaining a sample from the donated blood; isolating an alloantiby panel from the recipient; operably linking an isolated alloantibody from the panel to a first functionalized nanocrystal, wherein the functionalized nanocrystal comprises: a host molecule; a rare earth ion sensitizer; a rare earth ion emitter; a coordination ligand capping the rare-earth ion doped nanocrystal, wherein the coordination ligand forces a substantially pure phase on the nanocrystal; and a functionalizing coating, wherein the functionalizing coating does not affect the optical properties of the nanocrystal; contacting the donated sample with the functionalized nanocrystal operably linked to the recepient's alloantibody; exposing the blood sample contacted with the functionalized nanocrystal operably linked to the recepient's alloantibody, to an electromagnetic radiation source; and detecting an optical emission frequency, wherein the lower the intensity of the optical emission, the higher the match between the donated red blood cells and the recipient's.

In another embodiment, the present invention further provides a method of performing direct antiglobulin test (DAT) on a subject comprising the steps of: isolating erythrocytes from the subject; isolating an immunoglobulin G (IgG) antibody panel from the subject; operably linking the isolated immunoglobulin G (IgG) antibody from the panel to a first functionalized nanocrystal, wherein the functionalized nanocrystal comprises: a host molecule; a rare earth ion sensitizer; a rare earth ion emitter; a coordination ligand capping the rare-earth ion doped nanocrystal, wherein the coordination ligand forces a substantially pure phase on the nanocrystal; and a functionalizing coating, wherein the functionalizing coating does not affect the optical properties of the nanocrystal; contacting the erythrocytes with the functionalized nanocrystal operably linked to the subject's immunoglobulin G (IgG) antibody; exposing the blood sample contacted with the functionalized nanocrystal operably linked to the subject's immunoglobulin G (IgG) antibody, to an electromagnetic radiation source; and detecting an optical emission frequency, wherein the emission spectra indicates immunoglobulin attachment to the erythrocyte.

In another embodiment, the present invention further provides a method of functionalizing a rare-earth ion doped, upconverting nanocrystal, comprising the steps of coating a rare-earth ion doped, upconverting nanocrystal comprising: a host molecule; a rare earth ion sensitizer; a rare earth ion emitter; a coordination ligand capping the rare-earth ion doped nanocrystal, wherein the coordination ligand forces a substantially pure phase on the nanocrystal, with a functionalizing coating, wherein the functionalizing coating does not affect the optical properties of the nanocrystal.

Other features and advantages of the present invention will become apparent from the following detailed description examples and figures. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF FIGURES

FIG. 1: Excitation pathways for Er³⁺/Yb³⁺ and Tm³⁺/Yb³⁺ ion couples.

FIG. 2: A scheme of a blood bank testing procedure: no serum antibodies are detected.

FIG. 3: A scheme of a blood bank testing procedure: serum antibodies are detected.

FIG. 4: A scheme of a blood bank testing procedure: serum antibodies are detected—the positive signal.

FIG. 5: A scheme of phenotyping of red blood cells.

FIG. 6: A scheme of phenotyping of red blood cells with multiple antibodies.

FIG. 7: A scheme of a flow cytometry application.

FIG. 8: Micrographs of TEM of hexagonal Er-doped NCs approximately a) 10 nm, and b) 100 nm in size.

FIG. 9: Micrographs of TEM images of (A) NaYF4:Yb, Ho, (B) NaYF4:Yb,Tm; (C) their upconversion fluorescence spectra, and (D) a comparison of the emission spectra of NaYF4:Yb,Er NCs synthesized in TOPO compared with those synthesized in ODE/OA or OM.

FIG. 10: TEM images (left) of the SiO2 coated NaYF4:Yb,Er upconversion nanocrytals (enlarged at inset), and their emission spectra (right) before and after SiO2 coating.

FIG. 11: A photograph of NCs dispersable (left) in hexane, (middle) in ethanol after coating SiO2 layer, and (right) in pH=7.4 buffer after coating polymer layer.

FIG. 12: A graph showing the dependence of particle size on luminescent efficiency. The efficiency of quantum confined atom increases non-linearly with decreasing size from 7 nm to 2 nm

FIG. 13: Micrographs of human H460 lung cancer cell line incubated with 15 ug of photosensitizer conjugated nanoparticles with IR excitation OFF (left), and IR excitation ON (right). Uptake of the conjugate can be observed under the image on the right and correlated to appropriate cells on left hand image.

FIG. 14: RhD assay of red blood cells.

FIG. 15: Assay results for both positive and negative control and the normalized positive as well as the numerical readout for the assay

### DETAILED DESCRIPTION OF THE INVENTION

In one embodiment, the present invention provides a composition comprising an antibody and a rare-earth based crystal.

In another embodiment, the present invention provides a rare-earth ion doped, upconverting nanocrystal, comprising: a. a host molecule; b. a rare earth ion sensitizer; c. a rare earth ion emitter; and d. a coordination ligand capping the rare-earth ion doped nanocrystal. In another embodiment, the coordination ligand forces a substantially pure phase on the nanocrystal. In another embodiment, the coordination ligand forces a pure phase on the nanocrystal.

In another embodiment, the present invention further provides a functionalized rare-earth ion doped, upconverting nanocrystal comprising: a host molecule; b. a rare earth ion sensitizer; c. a rare earth ion emitter; and d. a coordination ligand capping the rare-earth ion doped nanocrystal, wherein the coordination ligand forces a substantially pure phase on the nanocrystal; and e. a functionalizing coating, wherein the functionalizing coating does not affect the optical properties of the nanocrystal. In another embodiment, functionalizing coating is performed with a layer of silica.

In another embodiment, the present invention provides a method of detecting the presence of an antibody that binds an antigen on a red blood cell, comprising the steps: contacting a first sample comprising a first antibody with a red blood cell; spinning followed by washing the first sample and the red blood cell resulting in a second sample; contacting the second sample with a secondary antibody linked to a rare-earth based crystal, the secondary antibody specifically binds the first antibody; spinning followed by washing the second sample and the secondary antibody resulting in a third sample; and irradiating the third sample, wherein a presence of an antibody that binds an antigen on a red blood cell results in a positive signal, thereby detecting the presence of an antibody that binds an antigen on a red blood cell.

In another embodiment, the present invention provides a method of determining compatibility between a blood sample and a recipient of a blood sample, wherein the donor of the blood sample and the recipient are the same specie, comprising the steps: contacting serum of the recipient with a red blood cell from the blood sample; spinning followed by washing the serum and the red blood resulting in a first sample; contacting the first sample with a secondary antibody linked to a rare-earth based crystal, the secondary antibody specifically binds the specie antibodies; spinning followed by washing the first sample and the secondary antibody resulting in a second sample; and irradiating the second sample, wherein a presence of an antibody in the serum that binds an antigen on the red blood cell results in a positive signal indicating incompatibility between the blood sample and the recipient, thereby determining compatibility between a blood sample and a recipient of a blood sample.

In another embodiment, the present invention provides a method of phenotyping red blood cells, comprising the steps: contacting the red blood cells with at least one primary antibody against a first red blood cell antigen; spinning followed by washing the red blood cells and the at least one primary antibody against a first red blood cell antigen resulting in a first sample; contacting the first sample with a secondary antibody linked to a rare-earth based crystal, the secondary antibody specifically binds the at least one primary antibody; spinning followed by washing the first sample and the secondary antibody resulting in a second sample; and irradiating the second sample, wherein a positive signal indicates a presence of at least one red blood cell antigen on the red blood cells, thereby phenotyping red blood cells.

In another embodiment, the present invention provides a method of phenotyping red blood cells, comprising the steps: contacting the red blood cells with at least one antibody against a first red blood cell antigen, wherein the antibody is linked to a first rare-earth based crystal; spinning followed by washing the red blood cells and at least one antibody resulting in a first sample; irradiating the first sample, wherein a positive signal indicates a presence of a first red blood cell antigen on the red blood cells, thereby phenotyping red blood cells.

In another embodiment, the present invention provides a method of isolating a cell population comprising a common antigen comprising the steps: contacting a mixed cell population comprising a cell population comprising a common antigen with an antibody against the common antigen, wherein the antibody is linked to a rare-earth based crystal; spinning followed by washing mixed cell population and the antibody resulting in a first sample; sorting the cell population comprising a common antigen with a laser cell sorter, wherein the laser cell sorter irradiates the mixed cell population and identifies the antibody linked to a rare-earth based crystal bound to a cell, thereby isolating a cell population comprising a common antigen.

In one embodiment, provided herein a composition comprising an antibody and a rare-earth based crystal. In another embodiment, a rare-earth based crystal is a crystal with a rare earth atom disposed in it. In another embodiment, a rare-earth based crystal is a nanoparticle. In another embodiment, a rare-earth based crystal is a nanocrystal (NC). In another embodiment, a rare-earth based crystal absorbs short wavelengths and emits longer wavelengths. In another embodiment, a rare-earth based crystal absorbs long wavelengths and emits short wavelengths.

In another embodiment, a rare-earth based crystal is ‘tunable’. In another embodiment, rare-earth based crystal illumination spectrum is a narrow band laser source. In another embodiment, the source is a broad band source. In another embodiment, the source is a LED. In another embodiment, the source is an atomic line lamp.

In another embodiment, a rare-earth based crystal is characterized by emission spectrum that is virtually independent from illumination. In another embodiment, a rare-earth based crystal emission wavelength is selected over a wide range throughout the x-ray, ultraviolet, visible, and infrared parts of the optical spectrum.

In another embodiment, a rare-earth based crystal emits from as little as femtoseconds to as long as milliseconds or longer. In another embodiment, a rare-earth based crystal is contaminated by a range of naturally emitting species that can limit detection efficiency. In another embodiment, a rare-earth based crystal is routinely differentiated from background noise by utilizing custom emission lifetime of the rare-earth based crystal and the simple existing temporally differentiating detection technologies.

In another embodiment, provided herein a combination of rare earth crystals with antibodies and testing for light reaction after antibodies have been bound and incubated. In another embodiment, positive light (positive signal) reaction indicate binding of antibodies to target antigen.

In another embodiment, a rare earth crystal comprises upconverting phosphors in which rare earth atoms are embedded in a crystalline matrix. In another embodiment, a rare earth crystal absorbs infrared radiation and upconvert to emit in the visible spectrum through a series of real as opposed to virtual levels as in conventional two-photon dyes. In another embodiment, upconversion mechanism is a sequential excitation of the same atom or excitation of two centers and subsequent energy transfer. In another embodiment, emission of upconverting phosphors consists of sharp lines characteristic of atomic transitions in a well-ordered matrix.

In another embodiment, provided herein the use of different rare earth dopants. In another embodiment, different rare earth dopants comprise a large number of distinctive emission spectra. In another embodiment, a rare earth crystal comprises high IR-visible conversion cross section thus making it virtually background-free marker.

In another embodiment, a rare earth crystal reduces bleaching. In another embodiment, a rare earth crystal is not toxic. In another embodiment, a rare earth crystal is a rare earth oxide.

In another embodiment, a rare earth crystal is prepared by homogeneous precipitation.

In another embodiment, the rare earth crystal is NaYF₄ doped with Yb, Ln (Ln=Er, Ho and Tm) upconversion nanocrystals. In another embodiment, NaYF₄ doped with Yb, Ln (Ln=Er, Ho and Tm) upconversion nanocrystals is synthesized in trioctylphosphine oxide (TOPO) solvent via a thermolysis method. In another embodiment, NaYF₄ doped with Yb, Ln (Ln=Er, Ho and Tm) upconversion nanocrystals is synthesized in oleic acid. In another embodiment, the rare earth crystal is in cubic-phase. In another embodiment, the rare earth crystal is in hexagonal-phase. In another embodiment, the rare earth crystal is coated with SiO2 using microemulsion reaction. In another embodiment, the rare earth crystal is coated with SiO2 using microemulsion reaction for further biofunctionalization in antibody based applications.

In another embodiment, the rare earth crystal is a rare-earth doped yttria upconversion nanophosphor. In another embodiment, a rare-earth doped yttria upconversion nanophosphor is synthesized using a single-step gas-phase flame synthesis method. In another embodiment, the phosphors are characterized by x-ray diffractometry, transmission electron microscopy, and fluorescence spectroscopy. In another embodiment, particle size, morphology, and photoluminescence intensity are strongly affected by flame temperature. In another embodiment, gas-prepared nanophosphors are mostly single crystallites with an average size less than 30 nm.

In another embodiment, a rare-earth based crystal is about 1 nm to 50 nm In another embodiment, a rare-earth based crystal is about 1 nm to 5 nm. In another embodiment, a rare-earth based crystal is about 3 nm to 6 nm. In another embodiment, a rare-earth based crystal is about 5 nm to 10 nm. In another embodiment, a rare-earth based crystal is about 8 nm to 12 nm. In another embodiment, a rare-earth based crystal is about 12 nm to 20 nm. In another embodiment, a rare-earth based crystal is about 15 nm to 25 nm In another embodiment, a rare-earth based crystal is about 20 nm to 30 nm In another embodiment, a rare-earth based crystal is about 30 nm to 40 nm In another embodiment, a rare-earth based crystal is about 40 nm to 50 nm.

In another embodiment, a rare-earth based crystal is about 50 nm to 250 nm In another embodiment, a rare-earth based crystal is about 40 nm to 80 nm In another embodiment, a rare-earth based crystal is about 80 nm to 120 nm. In another embodiment, a rare-earth based crystal is about 100 nm to 200 nm. In another embodiment, a rare-earth based crystal is about 170 nm to 250 nm

### Lanthanide ion Doped NIR-Upconverting Nanocrystals

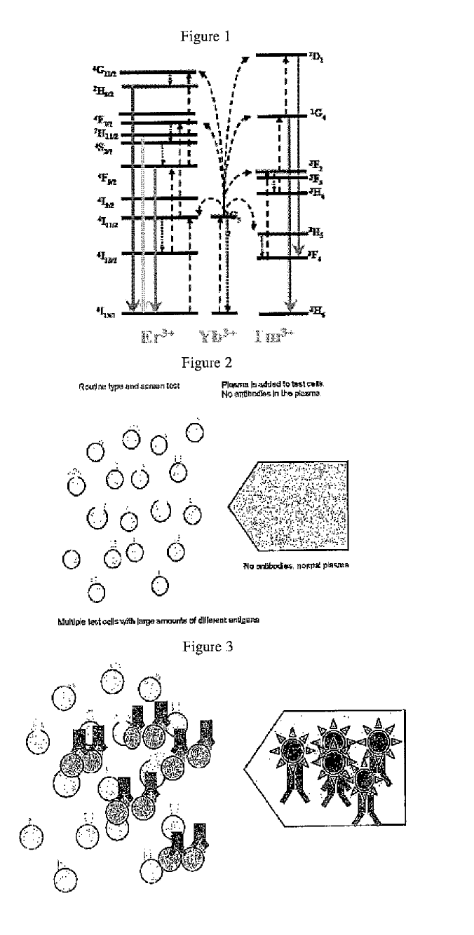

In another embodiment, lanthanide ion doped upconverting phosphor nanocrystals (NCs) convert two or more photons of lower energy (e.g. 980 nm) into one higher energy photon in the visible light range. In another embodiment, the antibodies as described herein are labeled with these compounds. In another embodiment, the antibodies as described herein are linked to these compounds. In another embodiment, NaYF₄: 2% Er³⁺, 20% Yb³⁺, is used with an initial energy transfer from an Yb³⁺ ion in the 2F_{5/2} state to an Er³⁺ ion populates the 4I_{11/2} level. In another embodiment, a second 980 nm photon, or energy transfer from an Yb³⁺ ion, populates the 4F_{7/2} level of the Er³⁺ ion. In another embodiment, the Er³⁺ ion is relax nonradiatively (without emission of photons) to the 2H_{11/2} and 4S_{3/2} levels, and the green 2H_{11/2}→4I_{15/2} and 4S_{3/2}→4I15/2 emissions occur. In another embodiment, the ion further relaxes and populates the 4F_{9/2} level leading to the red 4F_{9/2}→4I_{15/2} emission. In another embodiment, the 4F_{9/2} level is populated from the 4I_{13/2} level of the Er³⁺ ion by absorption of a 980 nm photon, or energy transfer from an Yb³⁺ ion, with the 4I_{13/2} state being initially populated via the nonradiative 4I_{1/2}→4I_{13/2} relaxation. In another embodiment, the NaYF₄: 2% Tm³⁺, 20% Yb³⁺ sample, up to four subsequent energy transfers from Yb³⁺ ions populate the upper Tm³⁺ levels and the various emissions can occur (see FIG. 1. for Excitation pathways for Er³⁺/Yb³⁺ and Tm³⁺/Yb³⁺ ion couples).

In another embodiment, rare earth phosphors convert near-infrared excitation wavelengths into a wide range of emission wavelengths in the visible spectrum. In another embodiment, rare earth phosphors compared to conventional fluorophores, have narrow emission bands, do not suffer from interference from autofluorescence or from photobleaching, and can be measured using relatively inexpensive detection equipment.

In another embodiment, efficient upconversion luminescence, comprises selection of an efficient host material with less non-radiative energy losses to accommodate lanthanide ions. In another embodiment, NaYF₄ matrixes, owing to its low vibrational energies and high ionicity, which lead to the minimum non-radiative quenching of the excited state of the rare earth ions, are used. In another embodiment, NaYF₄ hosts of lanthanide NCs occurs in either alpha-phase (cubic) or Beta-phase (hexagonal) crystals. In another embodiment, Beta-phase (hexagonal) crystals are used as they exhibit 20-30 times higher upconverting efficiency than that of alpha-phase at similar crystal sizes.

In another embodiment, synthesis of colloidal NaYF₄ hosts doped with rare earth lanthanides by thermolysis of lanthanide and trifluoroacetic precursors in high boiling point solvents, including oleic acid (OA), oleylamine, and octadecene (ODE) is preformed. In another embodiment, the coordination ligands cap the NCs, and prevent them from agglomeration during crystal growth and nucleation at high temperature.

In another embodiment, two novel ligand systems, OA/TOP (trioctylphosphine) and trioctylphosphine oxide (TOPO) in co-thermolysis synthesis method for manufacturing NaYF₄ hosted upconverting NCs are provided. In another embodiment, these solvents are used for the synthesis of colloidal lanthanide-doped upconverting NCs. In another embodiment, synthesis in TOPO at above 330° C. produced pure beta-phase NCs of small diameter (approximately 10 nm) with narrow size distribution (standard deviation of 0.73), and in OA/TOP, hexagonal-cylinder nanocrystals with uniform length/width/height are tunable from 50 nm to 200 nm In another embodiment, hydrophobic beta-phase NaYF₄:Yb, Ln NCs are made hydrophilic by coating them with a suitable material (e.g. silica or a polymer), paving the way for functionalization with reactive groups such as amines In another embodiment, coated upconverter NCs retain their unique optical properties. In another embodiment, coated NCs are coupled to antibodies as described herein. In another embodiment, coated NCs are coupled to secondary antibodies as described herein. In another embodiment, coated NCs are coupled to anti-RBC antibodies, and used for a range of diagnostic and research purposes.

In another embodiment, an antibody-linked crystal as described herein is 5-40 nm-diameter nanophosphor crystal. In another embodiment, an antibody-linked crystal as described herein is 10-30 nm-diameter nanophosphor crystal. In another embodiment, an antibody-linked crystal as described herein is 25-35 nm-diameter nanophosphor crystal. In another embodiment, an antibody-linked crystal as described herein is 20 nm-diameter nanophosphor crystal. In another embodiment, an antibody-linked crystal as described herein possess narrower emission bands than type-II quantum dots (QDs), aiding the simultaneous tracking of multiple red blood cell surface antigens. In another embodiment, an antibody-linked crystal as described herein is a visible light nanophosphor crystal. In another embodiment, a nanophosphor crystal possesses an irradiance larger than an equivalent emission wavelength 30 nm quantum dot that bears an organic biocompatible coating and an appropriate antibody-based-targeting agent. In another embodiment, the combination of narrow emission bands, adaptable emission wavelengths, and high irradiances compared with other nanoscale reporter particles, suggest that functionalized NIR up-converting nanophosphor crystals provide ultra-sensitive detection and multiplexed detection of a wide range of biological application based on antigen recognition. In another embodiment, the combination of narrow emission bands, adaptable emission wavelengths, and high irradiances compared with other nanoscale reporter particles, suggest that functionalized NIR up-converting nanophosphor crystals provide ultra-sensitive detection and multiplexed detection of a wide range of RBC antigens important in pre-transfusion blood-typing.

In another embodiment, advantages of rare-earth upconversion NCs compared with conventional down-conversion inorganic fluorophores, organic dyes, and semiconductor quantum dots include: inexpensive and high-power near infrared diode lasers as excitation source to produce visible luminescence, much higher signal-to-noise due to the absence of autofluorescence and reduction of light scattering, inherent resistance to photo-bleaching and photochemical degradation, and wide spectral domain and narrow emission output.

In another embodiment, provided herein a composition comprising antibody reagents, labeled with novel rare-earth NC reporters. In another embodiment, these reagents enable comprehensive typing of RBCs for all clinically-relevant antigens in an automated, multiplexed assay. consumable reagents. In another embodiment, these reagents enable comprehensive pre-transfusion matching.

In another embodiment, provided herein a composition comprising photodynamic reporter. In another embodiment, the photodynamic reporter is an antibody labeled with a rare-earth phosphor nanocrystal. In another embodiment, the photodynamic reporter is a rare-earth phosphor nanocrystal linked to an antibody. In another embodiment, the photodynamic reporter is a rare-earth phosphor nanocrystal attached to an antibody. In another embodiment, the photodynamic reporter is a rare-earth phosphor nanocrystal conjugated to an antibody. In another embodiment, the photodynamic reporter is a rare-earth phosphor nanocrystal chemically attached to an antibody.

In another embodiment, NCs synthesized via co-thermolysis method are capped with coordination ligands, which provide many advantages such as preventing agglomeration, controlling NC nucleation and growth and improving monodisperse etc. In another embodiment, the synthesis mechanism, surface modification transferring hydrophobic to hydrophilic and following antibody conjugation are similar to those used previously in quantum dots, which substantially mitigates the risk to produce final products.

In another embodiment, functionalization is performed by silica coating. In another embodiment, functionalization is performed by a silica-amine strategy. In another embodiment, functionalization is performed by polymeric coating using polyacrylic acid (PAA) which introduces carboxyl functional groups, which make hydrophobically ligated nanophosphors water-soluble and possible for further bioconjugation. In another embodiment, introducing the amino group onto amphiphilic-PAA coated NCs is carried out by reacting with cross-linker of EDC and further reacting with lysine. In another embodiment, after attaching amine groups, the antibodies are added by EDC-mediated coupling reaction. In another embodiment, functionalization is performed by ligand exchange, in which TOPO or OA/TOP ligands are replaced with thiodiglycolic acid to introduce carboxyl groups. In another embodiment, this approach is widely used on quantum dots.

In another embodiment, verification of binding of antibody is assessed using standard techniques (binding the conjugate antigen-antibody with antihuman IgG).

In another embodiment, an alternative method of detection, measuring infrared decay time is tailored for unique emissive properties. In another embodiment, at a minimum size of 8 nm the nanocrystal visible emission is detected with conventional spectrometry for a single particle.

In another embodiment, a solution to low emission intensity for smaller crystals is overcome by utilization of a technique recently described called single atom quantum confinement (Bhargava 2000). In another embodiment, this describes the ability of using a single atom emitter embedded in the crystalline matrix to give greater emission intensity when compared to 2-8 nm size particles with numerous emitters in the center (FIG. 12).

In another embodiment, the crystal is linked to an antibody. In another embodiment, the antibody is a polyclonal antibody. In another embodiment, the antibody is a monoclonal antibody. In another embodiment, the antibody is a monospecific antibody. In another embodiment, the antibody is a single-chain variable fragment (SCfV) antibody.

In another embodiment, the antibody binds a ligand in a cell. In another embodiment, the antibody binds a ligand displayed on a cell membrane. In another embodiment, the ligand is a protein. In another embodiment, the ligand is a glycoprotein. In another embodiment, the ligand is a receptor. In another embodiment, the ligand is an adhesion protein. In another embodiment, the ligand is a hormone. In another embodiment, the ligand is a cytokine. In another embodiment, the ligand is a chemokine. In another embodiment, the terms “antibody binds” and “antibody recognizes” are used interchangebly.

In another embodiment, the composition comprises a stabilizer. In another embodiment, the stabilizer is a protein. In another embodiment, the stabilizer is BSA. In another embodiment, the composition comprises sodium azide at concentrations ranging from 0.02 to 0.05% (w/v). In another embodiment, the composition comprises glycerol.

In another embodiment, the composition comprises a polyol. In another embodiment, the composition comprises a buffer such as acetate(e.g., sodium acetate, potassium acetate, magnesium acetate) and acetic acid (e.g., at a concentration of about 1 mM to about 20 mM) and sucrose (e.g., at a concentration of about 5 mg/ml to about 70 mg/ml). In another embodiment, the composition is at a pH of about 4.5 to about 7.0. In another embodiment, the composition is at a pH of about 5.5 to about 6.0.

In another embodiment, the composition is a stable aqueous formulation comprising a an effective amount of an antibody. In another embodiment, the composition is a stable aqueous formulation comprising a an effective amount of an antibody not subjected to prior lyophilizabon.

In another embodiment, the antibody is kept refrigerated. In another embodiment, the antibody is kept on ice. In another embodiment, the antibody is kept frozen. In another embodiment, the antibody is kept in a lyophilized powder.

In another embodiment, provided herein a method of detecting the presence of an antibody that binds an antigen on a red blood cell, comprising the steps: a) contacting a first sample comprising a first antibody with a red blood cell; b) spinning followed by washing the first sample and the red blood cell resulting in a second sample; c) contacting the second sample with a secondary antibody linked to a rare-earth based crystal, wherein the secondary antibody specifically binds the first antibody; d) spinning followed by washing the second sample and the secondary antibody resulting in a third sample; and e) irradiating the third sample, wherein a presence of an antibody that binds an antigen on a red blood cell results in a positive signal, thereby detecting the presence of an antibody that binds an antigen on a red blood cell. In another embodiment, provided herein a method of detecting the presence of an antibody that recognizes an antigen on a red blood cell, comprising the steps: a) contacting a first sample comprising a first antibody with a red blood cell; b) spinning to followed by washing the first sample and the red blood cell resulting in a second sample; c) contacting the second sample with a secondary antibody linked to a rare-earth based crystal, wherein the secondary antibody specifically binds the first antibody; d) spinning followed by washing the second sample and the secondary antibody resulting in a third sample; and e) irradiating the third sample, wherein a presence of an antibody that recognizes an antigen on a red blood cell results in a positive signal, thereby detecting the presence of an antibody that recognizes an antigen on a red blood cell.

In another embodiment, red blood cells are transfused as type specific red blood cells, i.e. a group A patient receives group A blood. In another embodiment, a patient with Rhesus positive groups, receives RH+ blood. In another embodiment, patients develop antibodies against red blood cells which are different from antibodies agains A or B. In another embodiment, antibodies may develop after previous transfusions, after childbirth etc. In another embodiment, provided herein a method for detecting the presence of such antibodies in a blood sample. In another embodiment, provided herein a method for testing for presence of those “unexpected” antibodies in a test called type and screen.

In another embodiment, patient's red blood cells are tested for blood group and type, i.e. A, B or O group and Rh type and the patient's plasma is tested for presence of unexpected antibodies' (screen).

In another embodiment, provided herein a method of screening an antibody against a red blood cell antigen. In another embodiment, provided herein a method of screening a recombinant antibody against a red blood cell antigen. In another embodiment, provided herein a method of screening a Scfv against a red blood cell antigen. In another embodiment, provided herein a method of screening a monoclonal antibody against a red blood cell antigen. In another embodiment, provided herein a method of screening antisera against a red blood cell antigen.

In another embodiment, provided herein a method of determining compatibility between a blood sample and a recipient of a blood sample, wherein the donor of a blood sample and a recipient are of the same specie, comprising the steps: a) contacting serum of a recipient with a red blood cell from a blood sample; b) spinning followed by washing the serum and the red blood resulting in a first sample; c) contacting the first sample with a secondary antibody linked to a rare-earth based crystal, wherein the secondary antibody specifically binds the specie antibodies; d) spinning followed by washing the first sample and the secondary antibody resulting in a second sample; and e) irradiating the second sample, wherein a presence of an antibody in the serum that binds an antigen on the red blood cell results in a positive signal indicating incompatibility between the blood sample and the recipient, thereby determining compatibility between a blood sample and a recipient of a blood sample. In another embodiment, the blood sample is a blood sample obtained from a donor. In another embodiment, the donor is a first subject and the recepient is a second subject.

In another embodiment, provided herein a method of phenotyping red blood cells, comprising the steps: a) contacting a red blood cells with at least one primary antibody against a first red blood cell antigen;b) spinning followed by washing red blood cells and at least one primary antibody against a first red blood cell antigen resulting in a first sample; c) contacting a first sample with a secondary antibody linked to a rare-earth based crystal, wherein the secondary antibody specifically binds at least one primary antibody; d) spinning followed by washing a first sample and a secondary antibody resulting in a second sample; and e) irradiating a second sample, wherein a positive signal indicates a presence of at least one red blood cell antigen on red blood cells, thereby phenotyping red blood cells. In another embodiment, provided herein a method of phenotyping red blood cells, comprising the steps: a) contacting a red blood cells with a primary antibody against a red blood cell antigen;b) spinning followed by washing red blood cells and the primary antibody against a red blood cell antigen resulting in a first sample; c) contacting a first sample with a secondary antibody linked to a rare-earth based crystal, wherein the secondary antibody specifically binds the primary antibody; d) spinning followed by washing a first sample and a secondary antibody resulting in a second sample; and e) irradiating a second sample, wherein a positive signal indicates a presence of a red blood cell antigen on red blood cells, thereby phenotyping red blood cells.

In another embodiment, provided herein a method for the identifying the nature of an antibody bound to a patient's red blood cells. In another embodiment, provided herein a method for the identifying the nature of an antibody in the serum bound to a patient's red blood cells.

In another embodiment, provided herein an improved positive direct antiglobulin test (DAT). In another embodiment, provided herein a method for identifying a panreactive antibody. In another embodiment, provided herein a method for reducing the risk of a hemolytic transfusion reaction. In another embodiment, provided herein a method for pretransfusion compatibility testing includes an ABO and/or Rh type and antibody screen on the donor unit.

In another embodiment, provided herein an improved coombs test. In another embodiment, provided herein a method for identifying a disease or condition in an individual's blood that may contain IgG antibodies that can specifically bind to antigens on the red blood cell surface membrane, and their circulating red blood cells can become coated with IgG alloantibodies and/or IgG autoantibodies. In another embodiment, provided herein a method for reducing the risk of complement proteins activation. In another embodiment, provided herein a method to detect antibodies or complement proteins that are bound to the surface of red blood cells. In another embodiment, the method comprises a blood sample, wherein the red blood cells are washed treated with a sample comprising an antibody such as but not limited to serum and then incubated with an additional antibody comprising a rare earth crystal, wherein the additional antibody comprising a rare earth crystal recognizes or specifically binds a common antigen present on all antibodies present in a sample. In another embodiment, the method comprises washed red blood cells treated with a sample comprising serum and then incubated with an additional antibodywhich specifically recognizes or specifically binds a common antigen present on all antibodies present in a sample. In another embodiment, the method comprises washed human red blood cells treated with a sample comprising human serum and then incubated with an anti-human IgG1 antibody. In another embodiment, the method comprises washed human red blood cells treated with a sample comprising human serum and then incubated with an anti-human IgG1 antibody comprising a rare earth crystal. In another embodiment, the method comprises washed human recepient red blood cells treated with a sample comprising human donor serum and then incubated with an anti-human IgG antibody. In another embodiment, the method comprises washed human red blood cells treated with a sample comprising human serum and then incubated with an anti-human IgG1 antibody comprising a rare earth crystal.

In another embodiment, the donor and the recipient have the same blood type. In another embodiment, the donor and the recipient have the different blood type.

In another embodiment, phenotyping red blood cells comprises determination of red cell antigen phenotype. In another embodiment, phenotyping red blood cells comprises determination of red cell antigen phenotype of nonalloimmunized sickle cell disease. In another embodiment, phenotyping red blood cells comprises comprises ABO determination. In another embodiment, phenotyping red blood cells comprises D, C, E, and K antigens determination. In another embodiment, phenotyping red blood cells comprises Rh determination.

In another embodiment, a method of phenotyping red blood cells comprises contacting a red blood cells with number of different primary antibodies. In another embodiment, each of the different primary antibodies recognizes a different epitipoe on a red blood cell antigen. In another embodiment, each of the different primary antibodies recognizes a different epitipoe on a red blood cell antigen, present on the cytoplasmic membrane. In another embodiment, each of the different primary antibodies recognizes a different red blood cell antigen. In another embodiment, each of the different primary antibodies recognizes a different red blood cell antigen, present on the cytoplasmic membrane.

In another embodiment, a method of phenotyping red blood cells comprises contacting a first sample with at least one secondary antibody linked to a rare-earth based crystal, wherein each secondary antibody specifically binds one primary antibody. In another embodiment, a method of phenotyping red blood cells comprises contacting a first sample with at least one secondary antibody linked to a rare-earth based crystal, wherein each secondary antibody specifically binds one primary antibody and not other primary antibodies. In another embodiment, each primary antibody is produced in a different specie such as but not limited to: goat, rabbit, mouse, rat. In another embodiment, secondary antibody binding is based on unique epitope present in one primary antibody but not present in other primary antibodies. In another embodiment, secondary antibodies comprise: anti-goat IgG antibody, anti-mouse IgG antibody, anti-rat IgG, anti-rabbit IgG antibody etc. In another embodiment, a secondary antibody is designed to bind only one epitope present in one primary antibody. In another embodiment, each secondary antibody comprises a different rare-earth based crystal. In another embodiment, each rare-earth based crystal comprises different optical properties. In another embodiment, the different optical properties of each rare-earth based crystal provide means for identification of each antigen. In another embodiment, a method of phenotyping red blood cells as described herein provides identification of multiple antigens in a single reaction. In another embodiment, a method of phenotyping red blood cells as described herein provides accurate identification of multiple antigens in a single reaction.

In another embodiment, red blood cells are human red blood cells. In another embodiment, red blood cells are mammalian red blood cells. In another embodiment, red blood cells are rodent red blood cells. In another embodiment, red blood cells are pet red blood cells. In another embodiment, red blood cells are farm animal red blood cells. In another embodiment, red blood cells are monkey red blood cells. In another embodiment, red blood cells are mouse red blood cells. In another embodiment, red blood cells are human rabbit blood cells. In another embodiment, red blood cells are rat red blood cells. In another embodiment, red blood cells are guinea pig red blood cells.

In another embodiment, provided herein a method of phenotyping red blood cells, comprising the steps: a) contacting red blood cells with at least one antibody against a first red blood cell antigen, wherein the antibody is linked to a first rare-earth based crystal; b) spinning followed by washing the red blood cells and at least one antibody resulting in a first sample; c)irradiating the first sample, wherein a positive signal indicates a presence of a first red blood cell antigen on red blood cells, thereby phenotyping red blood cells. In another embodiment, at least one antibody against a first red blood cell antigen comprises first rare-earth based crystal. In another embodiment, at least one antibody against a first red blood cell antigen is linked to a first rare-earth based crystal. In another embodiment, an antibody as described herein is linked to a rare-earth based crystal.

In another embodiment, a method as described herein comprises two antibodies: a first or primary antibody that recognizes a red blood cell antigen and a secondary antibody that recognizes the first or primary antibody. In another embodiment, a method of phenotyping comprises two antibodies: a first or primary antibody that recognizes a red blood cell antigen and a secondary antibody that recognizes the first or primary antibody. In another embodiment, a method of phenotyping comprises two antibodies: a first or primary antibody that binds a red blood cell antigen and a secondary antibody that binds the first or primary antibody. In another embodiment, a method as described herein comprises two antibodies: a first or primary antibody that binds a red blood cell antigen and a secondary antibody that binds the first or primary antibody. In another embodiment, a method of phenotyping comprises two antibodies: a first or primary antibody that specifically binds a red blood cell antigen and a secondary antibody that specifically binds the first or primary antibody. In another embodiment, a method of phenotyping comprises two antibodies: a first or primary antibody that specifically binds a red blood cell antigen and a secondary antibody that binds the first or primary antibody. In another embodiment, a method as described herein comprises two antibodies: a first or primary antibody that specifically binds a red blood cell antigen and a secondary antibody that binds the first or primary antibody. In another embodiment, a secondary antibody as described herein is linked to a rare-earth based crystal.

In another embodiment, a method as described herein comprises a single antibody that recognizes a red blood cell antigen and is linked to a rare-earth based crystal. In another embodiment, a method as described herein comprises a single antibody that binds a red blood cell antigen and is linked to a rare-earth based crystal. In another embodiment, a method as described herein comprises a single antibody that specifically binds a red blood cell antigen and is linked to a rare-earth based crystal. In another embodiment, a method as described herein comprises a single antibody that recognizes a red blood cell antigen and comprises a rare-earth based crystal.

In another embodiment, a method of phenotyping comprises a single antibody that recognizes a red blood cell antigen and is linked to a rare-earth based crystal. In another embodiment, a method of phenotyping comprises a single antibody that binds a red blood cell antigen and is linked to a rare-earth based crystal. In another embodiment, a method of phenotyping comprises a single antibody that specifically binds a red blood cell antigen and is linked to a rare-earth based crystal. In another embodiment, a method of phenotyping comprises a single antibody that recognizes a red blood cell antigen and comprises a rare-earth based crystal.

In another embodiment, a method as described herein further comprises an additional antibody against a second red blood cell antigen, wherein the additional antibody is linked to a second rare-earth based crystal. In another embodiment, a method as described herein further comprises an additional antibody that specifically binds a second red blood cell antigen, wherein the additional antibody is linked to a second rare-earth based crystal. In another embodiment, a method of phenotyping further comprises an additional antibody against a second red blood cell antigen, wherein the additional antibody is linked to a second rare-earth based crystal. In another embodiment, a method of phenotyping further comprises an additional antibody that specifically binds a second red blood cell antigen, wherein the additional antibody is linked to a second rare-earth based crystal. In another embodiment, a first earth based crystal and a second rare-earth based crystal differ. In another embodiment, a first earth based crystal and a second rare-earth based crystal comprise different optical characteristics.

In another embodiment, an antibody against a first red blood cell antigen is derived from antiserum against a first red blood cell antigen. In another embodiment, an antibody against a second red blood cell antigen is derived from antiserum against a second red blood cell antigen. In another embodiment, an additional antibody against an additional red blood cell antigen is derived from antiserum against the additional red blood cell antigen.

In another embodiment, an antibody binding a first red blood cell antigen is derived from antiserum against a first red blood cell antigen. In another embodiment, an antibody binding a second red blood cell antigen is derived from antiserum against a second red blood cell antigen. In another embodiment, an additional antibody binding an additional red blood cell antigen is derived from antiserum against the additional red blood cell antigen. In another embodiment, an antibody specifically binding a first red blood cell antigen is derived from antiserum against a first red blood cell antigen. In another embodiment, an antibody specifically binding a second red blood cell antigen is derived from antiserum against a second red blood cell antigen. In another embodiment, an additional antibody specifically binding an additional red blood cell antigen is derived from antiserum against the additional red blood cell antigen.

In another embodiment, a red blood cell antigen is Rh (C, D, E, c, e, Cw). In another embodiment, a red blood cell antigen is Kell. In another embodiment, a red blood cell antigen is Duffy. In another embodiment, a red blood cell antigen is Kidd. In another embodiment, a red blood cell antigen is Lewis. In another embodiment, a red blood cell antigen is MNS. In another embodiment, a red blood cell antigen is P. In another embodiment, a red blood cell antigen is D. In another embodiment, a red blood cell antigen is C. In another embodiment, a red blood cell antigen is E. In another embodiment, a red blood cell antigen is c. In another embodiment, a red blood cell antigen is e. In another embodiment, a red blood cell antigen is f. In another embodiment, a red blood cell antigen is V. In another embodiment, a red blood cell antigen is Cw. In another embodiment, a red blood cell antigen is Ce. In another embodiment, a red blood cell antigen is cE. In another embodiment, a red blood cell antigen is Kell. In another embodiment, a red blood cell antigen is Cellano. In another embodiment, a red blood cell antigen is Sutter. In another embodiment, a red blood cell antigen is Matthews. In another embodiment, a red blood cell antigen is Penney. In another embodiment, a red blood cell antigen is Rautenberg. In another embodiment, a red blood cell antigen is Duffy a. In another embodiment, a red blood cell antigen is Duffy b. In another embodiment, a red blood cell antigen is Kidd a. In another embodiment, a red blood cell antigen is Kidd b. In another embodiment, a red blood cell antigen is Lewis a. In another embodiment, a red blood cell antigen is Lewis b. In another embodiment, a red blood cell antigen is Lutheran a. In another embodiment, a red blood cell antigen is Lutheran b. In another embodiment, a red blood cell antigen is Colton a. In another embodiment, a red blood cell antigen is Colton b. In another embodiment, a red blood cell antigen is Dombrock a. In another embodiment, a red blood cell antigen is Dombrock b.

In another embodiment, a positive signal comprises a rare-earth based crystal emission in a predefined wavelength. In another embodiment, a positive signal comprises irradiating a rare-earth based crystal in a predefined wavelength. In another embodiment, irradiating is laser irradiation.

In another embodiment, a laser is a gas laser. In another embodiment, a laser is a helium-neon laser (HeNe). In another embodiment, a laser is a carbon dioxide laser. In another embodiment, a laser is an argon-ion laser. In another embodiment, a laser is a nitrogen transverse electrical discharge in gas at atmospheric pressure (TEA) laser. In another embodiment, a laser is a metal ion laser. In another embodiment, a laser is a helium-silver (HeAg) laser. In another embodiment, a laser is a neon-copper (NeCu) laser.

In another embodiment, a laser is a chemical laser. In another embodiment, a laser is a hHydrogen fluoride laser. In another embodiment, a laser is a deuterium fluoride laser.

In another embodiment, a laser is an excimer laser. In another embodiment, a laser is a F2 laser. In another embodiment, a laser is a solid-state laser. In another embodiment, a laser is a Nd:YAG laser. In another embodiment, a laser is a ruby laser. In another embodiment, a laser is a fiber laser. In another embodiment, a laser is a semiconductor laser laser. In another embodiment, a laser is a solid-state laser. In another embodiment, a laser is a Neodymium laser. In another embodiment, a laser is a Nd:YVO4 laser. In another embodiment, a laser is a Nd:YLF laser. In another embodiment, a laser is a Yb:KGW laser. In another embodiment, a laser is a Yb:SYS laser. In another embodiment, a laser is a BOYS laser. In another embodiment, a laser is a Yb:CaF2 laser. In another embodiment, a laser is a Yb:KYW laser. In another embodiment, a laser is a Ti:sapphire laser. In another embodiment, a laser is a photonic crystal laser. In another embodiment, a laser is a silicon laser. In another embodiment, a laser is a dye laser. In another embodiment, a laser is a free electron laser.

In another embodiment, the sample comprises serum. In another embodiment, the sample comprises saliva. In another embodiment, the sample comprises tears. In another embodiment, the sample comprises breast milk In another embodiment, the sample comprises B-cells. In another embodiment, the sample is a whole blood sample.

In another embodiment, a red blood cell is derived from a first subject and a first antibody is derived from a second subject. In another embodiment, a red blood cell and a first antibody is derived from one subject. In another embodiment, a first subject and a second subject are the same specie. In another embodiment, a first subject and a second subject are both human subjects. In another embodiment, a first subject, a second subject, is a mammal. In another embodiment, a first subject, a second subject, is a rodent. In another embodiment, a first subject, a second subject, is a mouse. In another embodiment, a first subject, a second subject, is a guinea pig. In another embodiment, a first subject, a second subject, is a rat. In another embodiment, a first subject, a second subject, is a rabbit. In another embodiment, a first subject, a second subject, is a dog. In another embodiment, a first subject, a second subject, is a monkey. In another embodiment, a first subject, a second subject, is a pet. In another embodiment, a first subject, a second subject, is a farm animal.

In another embodiment, the secondary antibody is an anti-first antibody IgG. In another embodiment, the secondary antibody is an anti-human IgG. In another embodiment, the secondary antibody is an anti-mouse IgG. In another embodiment, the secondary antibody is an anti-rat IgG. In another embodiment, the secondary antibody is an anti-rabbit IgG. In another embodiment, the secondary antibody is an anti-Scfv IgG. In another embodiment, the secondary antibody is an anti-rare earth crystal IgG. In another embodiment, the secondary antibody is an anti-mammal IgG. In another embodiment, a primary antibody is is derived from antiserum against a red blood cell antigen. In another embodiment, a primary antibody is a monoclonal antibody. In another embodiment, a primary antibody is is derived from antiserum against a red blood cell antigen. In another embodiment, a primary antibody is a polyclonal antibody. In another embodiment, the red blood cells are human red blood cells and the secondary antibody is an anti-human IgG. In another embodiment, the secondary antibody is anti-primary antibody IgG. In another embodiment, the secondary antibody is a monoclonal antibody anti-primary IgG.

In another embodiment, contacting a first sample with a red blood cell comprises incubating a first antibody and a red blood cell. In another embodiment, contacting the recipient's serum with a red blood cell comprises incubating the serum and the red blood cell. In another embodiment, contacting the recipient's serum with the donor's red blood cell comprises incubating the serum and the red blood cell. In another embodiment, contacting the first sample with a secondary antibody comprises incubating the first sample and the secondary antibody.

In another embodiment, contacting a first sample with a red blood cell comprises incubating a first antibody and a blood sample. In another embodiment, contacting a first sample with a red blood cell comprises incubating a first antibody and a biological sample comprising a red blood cell. In another embodiment, contacting a second sample with a secondary antibody comprises incubating a second sample and a secondary antibody. In another embodiment, contacting a first sample with a red blood cell comprises incubating the first sample and the red blood cell. In another embodiment, contacting red blood cells with at least one primary antibody against a first red blood cell antigen comprises incubating red blood cells with at least one primary antibody against a first red blood cell antigen. In another embodiment, contacting red blood cells with one primary antibody comprises incubating red blood cells with one primary antibody. In another embodiment, contacting red blood cells with a primary antibody against at least one red blood cell antigen comprises incubating red blood cells and the primary antibody.

In another embodiment, the term contacting as used herein comprises incubating. In another embodiment, the term incubating comprises allowing the binding of an antibody or a fragment thereof to an antigen. In another embodiment, the term incubating comprises allowing the binding of an antibody or a fragment thereof to an antigen present on a red blood cell.

In another embodiment, the term incubating comprises facilitating the binding of an antibody or a fragment thereof to an antigen. In another embodiment, the term incubating comprises facilitating the binding of an antibody or a fragment thereof to an antigen present on a red blood cell.

In another embodiment, the term incubating comprises facilitating the binding of a secondary antibody or a fragment thereof to an epitope present on a first antibody. In another embodiment, the term incubating comprises facilitating the binding of a secondary antibody or a fragment thereof to an antigen present in a primary antibody.

In another embodiment, incubating is preformed in an incubation chamber. In another embodiment, incubation period with antibody is 5-60 minutes. In another embodiment, incubation period with antibody is 1-2 hours. In another embodiment, incubation period with antibody is 3-5 hours. In another embodiment, incubation period with antibody is 4-6 hours.

In another embodiment, incubating is preformed at −20° C.-4° C. In another embodiment, incubating is preformed at −20° C.-0° C. In another embodiment, incubating is preformed at 0° C.-15° C. In another embodiment, incubating is preformed at −20° C.-−4° C. In another embodiment, incubating is preformed at 10° C.-20° C. In another embodiment, incubating is preformed at 20° C.-30° C. In another embodiment, incubating is preformed at 25° C.-40° C. In another embodiment, incubating is preformed at 30° C.-40° C. In another embodiment, incubating is preformed at 37° C. In another embodiment, incubating is preformed at 36° C. In another embodiment, incubating is preformed at 35° C. In another embodiment, incubating is preformed at 38° C. In another embodiment, incubating is preformed at 39° C. In another embodiment, incubating is preformed at 40° C. In another embodiment, incubating is preformed at 41° C. In another embodiment, incubating is preformed at 42° C.

In another embodiment, spinning followed by washing is repeated at least twice. In another embodiment, spinning followed by washing is repeated at least three times. In another embodiment, spinning comprises centrifugation at 500-1500×g for 3-12 minutes. In another embodiment, spinning comprises centrifugation at 600-100×g for 4-10 minutes. In another embodiment, spinning comprises centrifugation at 600-800×g for 7-9 minutes. In another embodiment, washing comprises washing the pellets. In another embodiment, washing comprises gently washing the pellets. In another embodiment, washing comprises pipetting up and down the pellet and wash buffer 1-10 times. washing the pellets. In another embodiment, washing comprises aspirating and/or discarding the supernatant after spinning In another embodiment, the steps of spinning followed by washing are known to one of average skill in the art.

In another embodiment, provided herein a method of isolating a cell population comprising a common antigen comprising the steps: a) contacting a mixed cell population comprising a cell population comprising a common antigen with an antibody against a common antigen, wherein the antibody is linked to a rare-earth based crystal; b) spinning followed by washing mixed cell population and the antibody resulting in a first sample; c) sorting the cell population comprising a common antigen with a laser cell sorter, wherein the laser cell sorter irradiates the mixed cell population and identifies an antibody linked to a rare-earth based crystal bound to a cell, thereby isolating a cell population comprising a common antigen.

In another embodiment, a mixed cell population is a whole blood sample. In another embodiment, a mixed cell population is a primary cell culture. In another embodiment, a mixed cell population comprises immune cells. In another embodiment, a mixed cell population comprises blood cells. In another embodiment, a mixed cell population comprises neoplastic cells. In another embodiment, a mixed cell population comprises stem cells.

In another embodiment, a cell population comprising a common antigen is a red blood cell population. In another embodiment, a cell population comprising a common antigen is a white blood cell population. In another embodiment, a cell population comprising a common antigen is an immune cell population. In another embodiment, a cell population comprising a common antigen is a B-cell population. In another embodiment, a cell population comprising a common antigen is a T-cell population. In another embodiment, a cell population comprising a common antigen is a stem cell population. In another embodiment, a cell population comprising a common antigen is a cancerous cell population. In another embodiment, a cell population comprising a common antigen is an epithelial cell population. In another embodiment, a cell population comprising a common antigen is a eukaryotic cell population.

In another embodiment, sorting a cell population comprising a common antigen with a laser cell sorter comprises irradiating a mixed cell population and identifying a rare-earth based crystal linked to an antibody which is bound to a cell. In another embodiment, the laser fitts the absorbing and emitting wavelengths of the rare-earth based crystal. In another embodiment, the laser cell sorter identifies a rare-earth based crystal emission in a predefined wavelength.

In another embodiment, the method of isolating a cell population based on an antigen expressed by a cell population as described herein can be used for any cell population and/or any antigen for which antibodies can be raised against.

In another embodiment, the method of isolating a cell population further comprises an additional antibody against a second antigen. In another embodiment, the method of isolating a cell population further comprises an additional antibody against a second red blood cell antigen. In another embodiment, an antibody as described herein is derived from antiserum against a cellular antigen. In another embodiment, an antibody as described herein is derived from antiserum against a red blood cell antigen. In another embodiment, an antibody as described herein is derived from antiserum against a membrane anchored antigen. In another embodiment, an antibody as described herein is derived from antiserum against a bacterial antigen. In another embodiment, an antibody as described herein binds a membrane receptor. In another embodiment, an antibody as described herein binds an adhesion molecule.

The following examples are presented in order to more fully illustrate the preferred embodiments of the invention. They should in no way be construed, however, as limiting the broad scope of the invention.

### EXAMPLES

### Example 1

### Blood Bank Testing for Presence of Antibodies

The screen test is performed as follows:

1. Two tubes with test red blood cells are prepared and the patient's serum is added.

2. The tubes are incubated, spun and washed a couple of times, resulting in a pellet of red blood cells.

a. If the patients serum contained NO antibodies, the test cells should be unaltered (FIG. 2).

b. If the patients serum contained antibodies, those antibodies are located on the surface of the test cells (FIG. 3).

3. Crystal bound Anti human IgG is added to the pellet/liquid and the specimen is incubated and washed a couple of times.

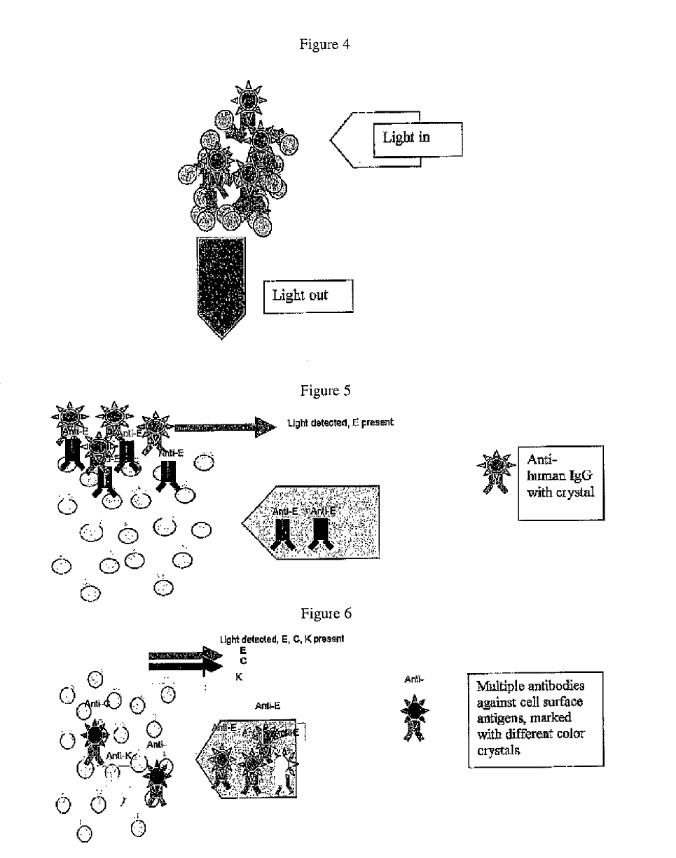

a. If the patients serum contained antibodies and the test cells are covered with patient's antibodies, then antihuman IgG binds to those antibodies and LIGHTS UP (positive signal) (FIG. 4).

b. If the patient's serum does not contain antibodies, the test cells remain unaltered and the anti-human IgG is washed away. NO LIGHTING occurs. The resulting product is reviewed with the light source and a positive light reaction is diagnosed as + antibody screen test (=serum antibodies present) or as negative light reaction − antibody screen (patient has no serum antibodies against the RBCs).

### Example 2

### Phenotyping Red Blood Cells (Primary and Secondary Antibodies)

Multiple tubes are prepared comprising patient's or donor's red blood cells. The specimen is washed to retain red blood cells only.

1. Addition of antiserum against one red blood cell antigen followed by incubation and washing a couple of times.

a. Red blood cells covered with antibodies result when red blood cells contain the antigen against which the antibody reacts.

b. If red blood cells do not contain the antigen against which the antibody reacts, the test cells remain unaltered.

2 A. Crystal associated Anti-human IgG is added to the pellet/liquid and the specimen is incubated and washed a couple of times.

a. Red blood cells covered with antibodies result when red blood cells contain the antigen against which the antibody reacts.

B. The Crystal associated anti-human IgG binds to those antibodies—a positive light reaction occurs (FIG. 5).

a. If red blood cells do not contain the antigen against which the antibody reacts, the test cells remain unaltered.

b. If red blood cells contain the antigen against which the antibody reacts, the crystal anti IgG binds and the positive complex and light it up (positive signal).

### Example 3

### Phenotyping Red Blood Cells (Single Antibody)

Instead of analyzing the cells with crystal associated anti-human IgG as secondary antibody multiple times for each surface antigen, the cells are analyzed by using multiple surface antibodies which are directly labeled with crystal associated antibodies.

Those antibodies react directly with antigen present on red blood cells. If the antibody reacts with the antigen, a positive light reaction occurs.

Multiple antibodies, each antibody directed to a different epitope and comprising a crystal with unique optical properties is used and analyzed (FIG. 6).

This allows for analysis of more than one antigen in one work process.

### Example 4

### Use of Flow Cytometry

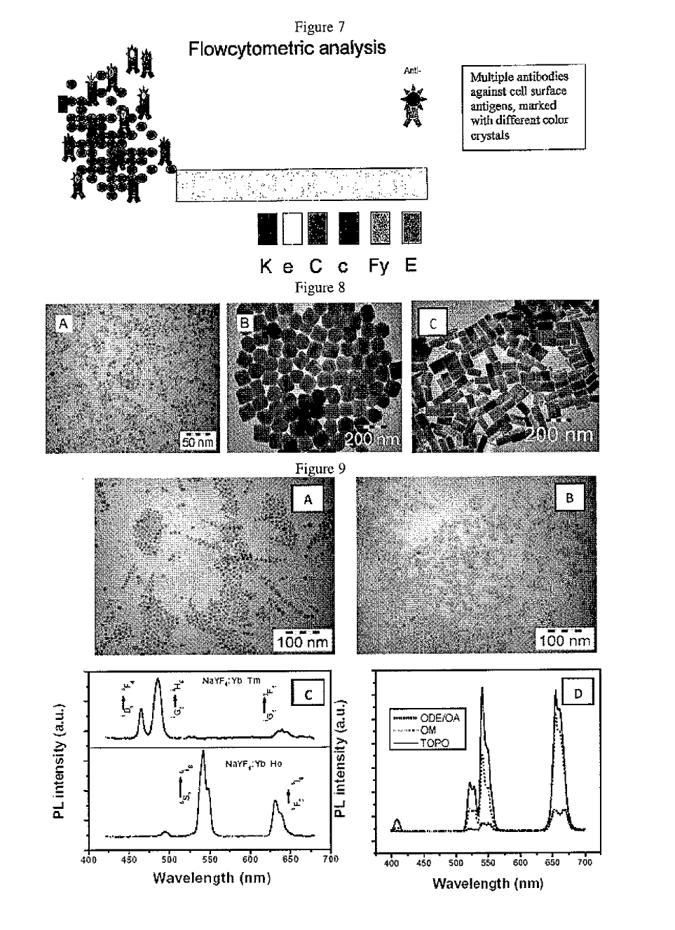

Antibodies against different red blood cell surface antigens arev used in a flow cytometry setting. With sensitive color reading, multiple colors and, thus, presence or absence of multiple antigens, cells are sorted, counted and analyzed(FIG. 7).

### Example 5

### Ln-Doped NaYF₄ Nanocrystals can be Synthesized by a Co-Thermolysis Method

A new ligand system was developed, namely TOPO and OA/ODE/TOP (ODE acted as non-coordination solvent and would not be capped on to final NCs), for manufacturing high efficiency up-converting Ln-doped β-phase NCs with narrow size distribution, efficient absorption in the infrared region, and discrete, quantifiable visible light emissions. To produce Er-, Ho-, or Tm-doped NCs, precursors (1.25 mmol CF₃COONa, 0.485 mmol (CF₃COO)₃Y, 0.25 mmol (CF₃COO)₃Yb, and (CF₃COO)₃Ln (Er, Ho, or Tm) were dissolved in 10 g TOPO or in OA/ODE/TOP (2:2:16 v/v/v). Under vigorous stirring in a 100 ml flask, the mixture was first heated to 100° C. under vacuum for 30 minutes to dehydrate the mixture, and then nitrogen was periodically purged through the solution. In the presence of nitrogen, the solution was rapidly heated to the reaction temperature (see below), and the reaction allowed to proceed for one hour. The reaction was allowed to cool and ethanol added to the cooled solution to precipitate the nanoparticles, which were isolated by centrifugation and washed in ethanol at least three times.

After introducing new ligand systems, synthesis in TOPO solvent could be conducted efficiently at a wide range of temperatures (280° C.-370° C., the optimal temperature being approximately 350° C.) and produced NCs around 10 nm; while synthesis in OA/ODE/TOP solvents, a new ligand was produced between OA and TOP, and this new ligand induced crystal phase transition which usually resulted in the β-phase NCs (hexagonal cylinders) with length of 100 nm, Transmission electron micrographs of NCs produced at 340° C. in TOPO (FIG. 8A) and 310° C. in OA/ODE/TOP (FIG. 8B) demonstrate the selective synthesis of pure β-phase NCs. Energy dispersive X-ray analysis (EDS) was applied to confirm the elemental compositions. X-ray diffraction (XRD, 30 kV and 20 mA, Cu Kα, Rigaku) patterns confirmed that crystals produced in both solvent systems were highly-pure high-efficiency β-phase. By carefully controlling reaction time, NC sizes were tunable from 5 nm to 20 nm in TOPO solvent, and from 50 nm to 200 nm in OA/ODE/TOP solvents in one single batch reaction. The reaction solution should be stirred vigorously to promise the more homogeneously distributed NCs. In addition, it was found that synthesis in OA/ODE/TOP solvents, rod-shape and hexagonal phase NCs could be obtained when increasing volume ratios of TOP over OA or by increasing CF₃COONa precursors (molar ratios of Na/Ln>2). TEM images of one example was shown in FIG. 8C. The results show that a method that can also control particle shape was developed too.

### Example 6

### Ln-Doped Upconversion Nanocrystals Synthesized in TOPO and OA/TOP/ODE Exhibit High-Quality Photoluminescent Properties

The photodynamic properties of Ln-doped NCs when synthesized using TOPO-protected co-thermolysis was investigated to ensure that NCs synthesized using this method retained a high-efficiency upconversion. Besides NaYF₄:Yb,Er NCs, NaYF₄:Yb,Ho and NaYF₄:Yb,Tm NCs of mean diameter 11 nm and 10 nm respectively were synthesized as described hereinabove (TEM images are shown in FIG. 9 A,B).

Photoluminescence measurements were performed at room temperature. A 980 nm laser diode (1 W maximum, Lasermate Group) was used as the excitation source and the beam was focused (12 cm focal length) to a 0.5 mm spot. The signals were focused to the end of an optical fiber and then delivered to the slit of a monochromater (SP-2500i, Princeton Instruments) with a 2400 g mm-1 grating (holographic, 400-700 nm). The signal was detected using a photomultiplier module (H6780-04, Hamamatsu Corp.) and was amplified by a lock-in amplifier (SR510, Stanford Research Systems) together with an optical chopper (SR540). The signal was recorded using the SpectraSense software data acquisition/analyser system (Princeton Instruments). FIG. 9C shows the emission spectra of NaYF4:Yb,Ho and NaYF4:Yb,Tm NCs. Although NCs synthesized in OA/TOP/ODE solvents also present highly efficient upconversion luminescence (not shown), those synthesized in TOPO solvents were substantially higher. FIG. 9D shows the emission spectrum of NaYF4:Yb,Er NCs synthesized in TOPO, compared with those synthesized in OA/ODE and OM solvents. Spectral bands corresponding to blue, green, and red emission transitions of theEr3+ are clearly depicted in the spectra. NCs synthesized from TOPO showed approximately 20 times higher emission intensity than those prepared from ODE/OA. This is due largely to the higher alpha to beta-phase efficiency of the TOPO technique.

### Example 7

### Ln-Doped Upconversion NCS can be made Hydrophilic by Silica and Amphiphilic Polymer Coating

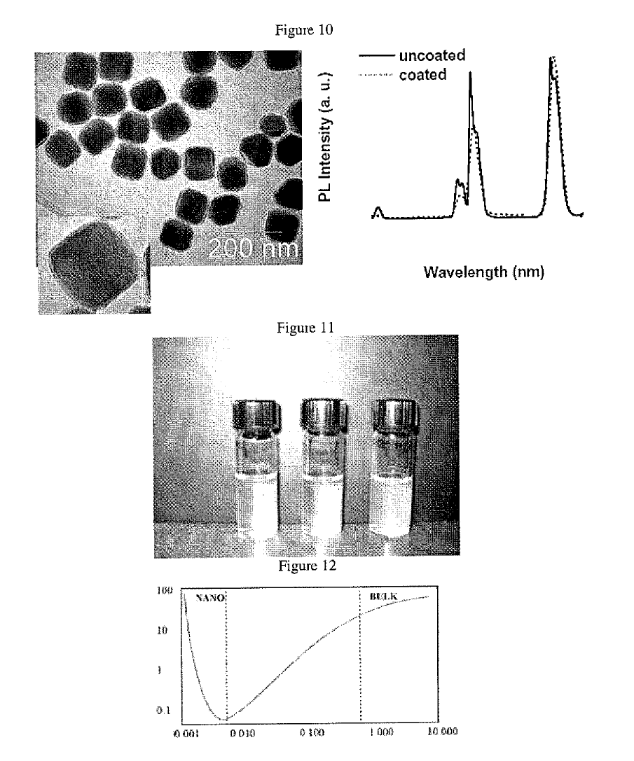

Because they are ligated by TOPO or OA/TOP, Ln-doped upconversion NCs are hydrophobic and exhibit high solubility in organic solvents such as hexane. However, for biological applications NCs need to be hydrophilic and soluble or dispersable in aqueous solvents. Two routes to transfer hydrophobic NCs to hydrophilic, silica coating and amphiphilic polymer coating were developed. Silica coating was applied to OA/TOP capped NCs, while amphiphilic polymer was coated onto both TOPO and OA/TOP capped NCs. OA/TOP capped NCs were coated with silica through reverse micelles via metal alkoxide hydrolysis and condensation in a micro-emulsion system (Darbandi, Lu et al. 2006). Briefly, hydrophobic NCs were dissolved in cyclohexane in which poly(ethylene glycol) nonylphenyl ether (NP-5) and tetraethoxysilane (TEOS) were added to form water-in-oil microemulsion. Dimethylamine was then added afterwards to hydrolyze TEOS and form SiO₂ layer onto NCs. The thickness of the SiO₂ layer was controlled by the reaction time and the amount of dimethylamine being added. In our work, the uniform SiO₂ layer can be controlled from 4 nm to 20 nm by varying above two factors (FIG. 10, left panel).

It was shown that a SiO2 layer thickness up to 8 nm (FIG. 10, left panel) did not affect luminescence intensity (FIG. 10, right panel). While with increasing the SiO2 layer, luminescence is accordingly decreased. After coating SiO2, NCs could be suspended in polar solvents such as ethanol for a period of time. Furthermore, these silica-coated NCs are suitable for further biofunctionalization (such as amine functionalization, as described in this proposal). OA/TOP- and TOPO-capped NCs were also coated with other polarizing and functional groups, including polyacrylic acid to introduce carboxyl groups for further biofunctionalization. Examples of NCs coated with OA/TOP, SiO2, and polyacrylic acid suspended in hexane, ethanol and pH=7.4 buffer, respectively, are shown in FIG. 11.

### Example 8

### Coating and Functionalization of NCS

In order to prepare for conjugation of the rare earth phosphor NCs with antibodies, a molecular coating is applied. Silica coating technique enables NCs to be functionalized with reactive amine groups. The silica coating method has been described previously. Briefly, for NCs capped with OA/TOP ligands a silica layer coating is applied by water in-oil (W/O) micro-emulsion containing NP-5 cyclohexane and water system. The NCs is dissolved in cyclohexane and mixed with surfactant NP-5 and tetraethoxysilane (TEOS). After stirring the mixture for 30 minutes, dimethylamine is added to hydrolyze the TEOS and form the silica layer. For hydrophilically ligated NCs, the NCs is suspended in isopropanol and mixed with TEOS directly; ammonia is used to hydrolyze the TEOS. The silica layer is observed under transmission electron microscopy and the thickness is calculated. Once a uniform silica coating has been added to NCs, reactive amine groups are added by mixing 3-aminopropyltrimethoxysilane (APS) with silica-coated NCs in ethanol. The presence of reactive amines on the silica-modified NCs are quantitatively confirmed using a ninhydrin-based procedure, in which primary amines react with ninhydrin to produce a color change. Amine coated NCs with minimum agglomeration are produced. Although the maximum amine groups on NCs facilitates the later bio-conjugation, adding too much APS causes unnecessary agglomeration. The optimum amount of APS along with the reaction conditions such as reaction temperature and time, is determined empirically.

### Example 9

### Conjugation of NCS to RBC Antibodies

Amine-functionalized NCs are cross-linked to glutaraldehyde by mixing amine coated NCs in buffer solution and then reacting them with streptavidin. Three proof-of-concept antibodies specific to erythrocyte antigens commonly tested for transfusion phenotyping (anti-D, anti-K, and anti-C) (Ortho Clinical Diagnostics, Raritan N.J.) are conjugated to the strepatvidin-linked NCs by performing an EDC (1-Ethyl-3-[3-dimethylaminopropyl]carbodiimide)-mediated coupling reaction in 10 mM borate buffer. The coupling reaction for each nanocrystal-antibody pair is performed twice, once using purified antibody, and once using FITC-conjugated antibody. The synthesized nanocrystal conjugates is purified by ultra-centrifugation. Concentrations of antibodies on NCs are determined experimentally combining with empirical calculation. Generally, total antibodies being detected are determined using a flow cytometer. By counting the average NC size and calculating the surface area per NC, evaluation of the number of antibodies on a single NC is given, then conjugation efficiency is determined

### Example 10

### Validation of the Stability and Binding of Rare-Garth Nanoparticle Functionalied Antibodies for Qualitative Determination of RBC Phenotype

The rare earth phosphor nanocrystal-conjugated antibodies are tested for the functionality of the antibody conjugates with regards to both their antigen binding and their photoluminescent properties. The influence of nanocrystal conjugation on antibody binding specificity is determined by analyzing binding to RBC samples obtained from the UPHS Presbyterian Hospital Blood Bank using flow cytometry. Because typical flow cytometers do not possess the ability to excite the NCs using infrared wavelengths, making detection of the crystals this way impossible, FITC-labeled nanocrystal-antibody conjugates are used. Having determined that conjugated antibodies are capable of binding RBC antigens satisfactorily, verification that the modification and functionalization of NCs does not influence their photo-emissive properties is performed. It is possible that these modifications may attenuate photon emission and influence the strength of the signal in aqueous solutions. The silica coating strategy described is a common functionalization strategy. Determination of the effect of the silica layer, amine groups, antibody conjugates, and (ultimately) the bound RBCs on the photo-emissive characteristics of the NCs is performed.

### Example 11

### Validation of Nanocrystal-Antibody Antigen Specificity

The ability of nanocrystal-conjugated antibodies to bind RBC antigens is determined by FACS. Red blood cells are diluted in saline to empirically-determined working concentrations and incubated for 15 minutes with nanocrystal-conjugated FITC-antibodies (NC-Ab-FITC). Binding is then analyzed using a Becton-Dickinson FACScan flow cytometer. Purified anti-human IgM is utilized as an isotype control. Forward- and side-scatter gates is set for intact RBC analysis, and FITC fluorescence is used to quantitate antibody binding. A range of different red blood cell samples is used sequentially, with varying degrees of antigen presentation, and therefore varying binding affinities as determined by the agglutination index performed previously at the blood bank and confirmed for standards. Indirect agglutination tests (IATs) is performed for direct and simultaneous comparison to the nanocrystal conjugates. In order to test the specificity, both homozygous and heterozygous test red blood cells are used to ensure that positive cells are detected, even with smaller amounts of antigen. Multiple commercially available test cell samples are used to verify the results. The combination of different antigens on red blood cells in the testing kits is chosen in such a way that these kits contain positive and negative controls. Each blood sample is tested with each of the antibodies (anti-K, anti-D, anti-C) labeled with each of the NCs (Er, Ho, Tm). For weakly-binding antibodies (anti-K, anti-D), a potentiator such as polyethylene glycol is added to prevent antibody shearing during the flow analysis. The binding of NC-Ab-FITC is compared in each case to unconjugated Ab-FITC controls. Replicate negative controls (cells known not to express the specific antigens) are tested to determine the threshold for positive reactions. Since some antibodies are weak binders, the sensitivity needs to be much greater (less than 5% deviation from controls).

### Example 12

### Validation of Photoemission Properties of Nanocrystal-Conjugated Antibodies

The ability to detect NC-Abs bound to RBCs using the spectral properties of the NCs themselves is determined Each sample of RBCs is suspended in HBSS, and allowed to bind to individual NC-Abs (each reagent at concentrations determined from the flow cytometry experiments, above). Unbound NC-Abs are removed by washing and the suspended RBCs placed in a 3 ml plastic cuvette and mounted in a fiber-optic absorption photometer jig (Ocean Optics). Nanophosphor excitation are achieved by illumination from a 980 nm solid state laser (Model BWF-5A, B&W Tek) collimated by the photometer jig via a 1 mm optical fiber. Emission is detected using a 400 micron optical fiber connected to a thermopile power detector (Model LM-45HTD, Coherent). Emission is verified by independent measurement after interrogation of each sample using a flat-cut fiber positioned above the cuvette and connected to a single-channel spectrograph (B&W Tek BTC112E) controlling for background and scatter. Negative controls comprise RBCs suspended in HBSS without NC-RBCs. The limit of sensitivity of each NC-Ab is determined In addition to testing the ability to detect individual NC-Abs bound to RBCs, RBCs bound to all three NC-conjugated antibodies (anti-IgG, anti-K, and antiD) are analyzed. The emission intensities of each antibody for each RBC sample is determined singly, and in combination stains, and compared to those determined by agglutination index, and by FACS (above). Relative binding affinities of those determined by FACS should be more sensitive to weaker binding antigens as well as antigens that are present in low quantities.

### Example 13

### Conjugated Nanoparticles Imaging

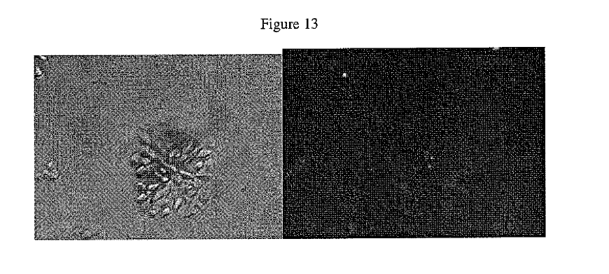

Conjugated nanoparticles prepared by Prud'homme, et al, were polymerized in Hanks Buffer by adding 5 mg/ml of PEG-PCL prior to incubation with H460, human lung cancer cells. The cells were incubated for 24 hours in 15 ug of PEG coated nanoparticle conjugate at 37° C., and 5% CO₂ atmosphere. Cells were washed twice in 1× Hanks buffer prior to imaging. The cells were imaged first under phase contrast imaging without IR excitation. Keeping the same cell group in view the field was exposed to 2.0 W/cm2 of 980 nm radiation and the image captured using a Hamamatsu Digital Imaging System. FIG. 13 depicts the cells with no infrared excitation and infrared excitation.

### Example 14

### RBC Phenotyping Utilizing Upconverting Nanocrystals

Inventors have successfully developed an assay capable of single particle detection for rapid blood phenotyping utilizing upconverting nanocrystals in a microfilter well format. For this study, inventors have constructed a nanocrystal conjugate with the absorption and emission of 980 nm and 540 nm, respectively.

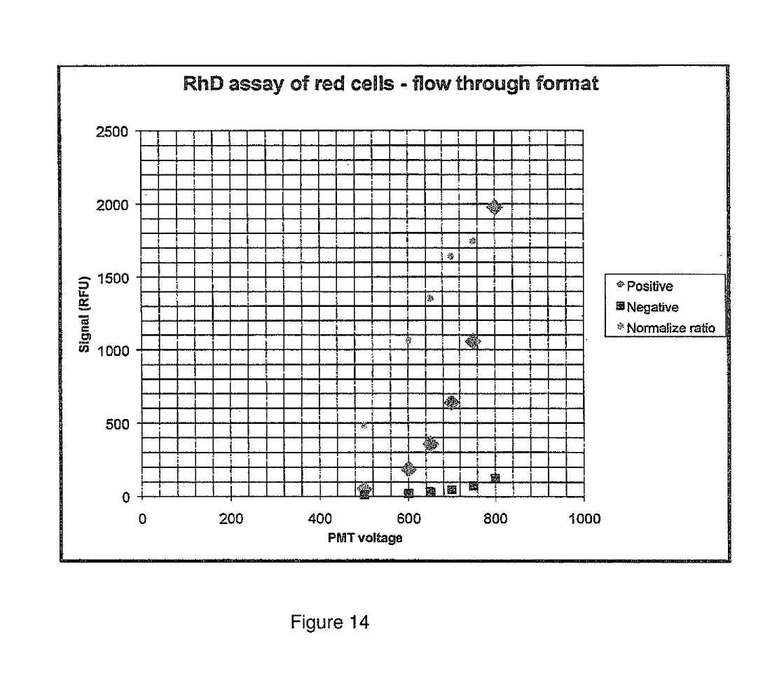

FIG. 14 shows RhD assay of red blood cells. Specifically, FIG. 14 shows the positive staining of anti-C antibody conjugated upconverters on human red blood cells positive with the C antigen. The negative controls showed little to no signal and the small amount of signal is attributed to slight background and agglomerated nanocrystals that may have been stuck in the filter. This problem has been rectified by performing a silica coating on the nanocrystals prior to antibody conjugation. The layer of silica can make the nanocrsytals more monodisperse and can prevent any aggregation so that they will not be trapped in the filter as well as neutralizing the surface charge on the nanocrystals preventing any non-specific binding. The degree of detection resolution that has been obtained is on the nanomolar scale. In addition, due to the ability of the nanocrystals to upconvert light from 980 nm to visible, the background signal is negligible allowing for single particle detection that would not be achieved utilizing conventional fluorophores and quantum dots. Nanocrystals of varying spectral signatures can be combined with other blood antibodies for a simultaneous, multiplexed readout. The flow through readout will also allow for multiple readout formats. An electronic optical platform has been designed to quantify the amount of positively bound nanocrystals. By isolating and tagging the positive red blood cells with upconverter using a 1 micron glass fiber filter the retained, labeled RBC's can also be visualized by the user by upconversion fluorescence microscopy.

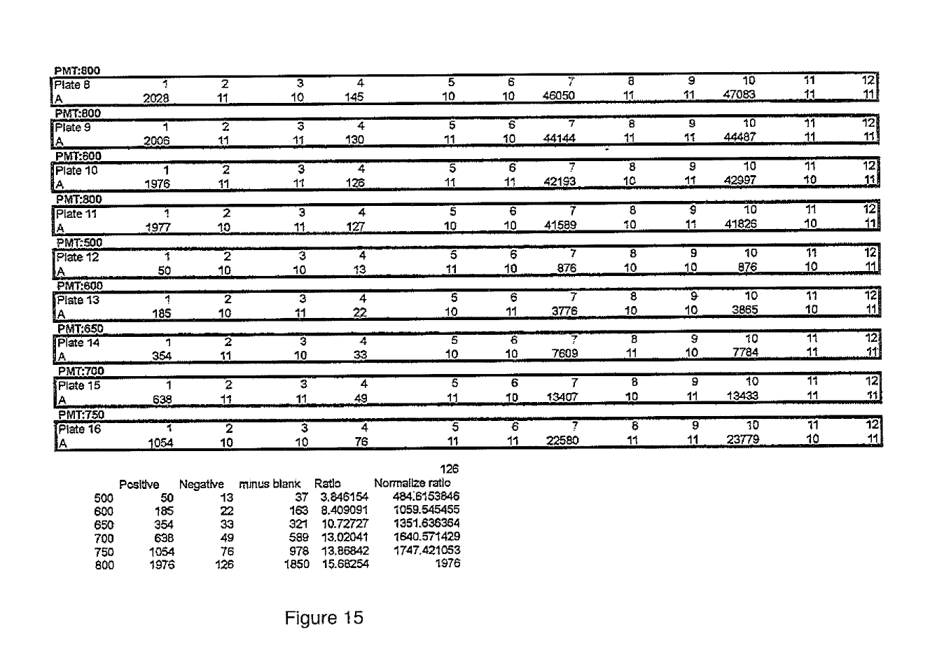

The data in FIGS. 14 and 15 show the assay results for both positive and negative control and the normalized positive as well as the numerical readout for the assay.

Having described preferred embodiments of the invention with reference to the accompanying drawings, it is to be understood that the invention is not limited to the precise embodiments, and that various changes and modifications may be effected therein by those skilled in the art without departing from the scope or spirit of the invention as defined in the appended claims.

What is claimed is:

1. A rare-earth ion doped, upconverting nanocrystal, comprising:
a. a host molecule;
b. a rare earth ion sensitizer;
c. a rare earth ion emitter; and
d. a coordination ligand capping the rare-earth ion doped nanocrystal,
wherein the coordination ligand forces a substantially pure phase on the nanocrystal.

2. The nanocrystal of claim 1, whereupon excitation with an electromagnetic source, the nanocrystal emits an optical energy having a frequency that exceeds the excitation frequency.

3. The nanocrystal of claim 1, wherein the rare-earth ion sensitizer or emitter is a lanthanide ion combination.

4. The nanocrystal of claim 1, wherein the coordination ligand is: an oleic acid/trioctylphosphine (OA/TOP) combination, or trioctylphosphine (TOPO).

5. The nanocrystal of claim 1, wherein the substantially pure phase of the nanocrystal is a β-phase.

6. The nanocrystal of claim 3, wherein the lanthanide is Lanthanum (La), Cerium (Ce), Praseodymium (Pr), Neodymium (Nd) Promethium (Pm), Samarium (Sm), Europium (Eu), Gadolinium (Gd), Terbium (Tb) Dysprosium (Dy), Holmium (Ho), Erbium (Er), Thulium (Tm), Ytterbium (Yb), or Lutetium (Lu).

7. The nanocrystal of claim 1, wherein the host molecule is a fluoride-based or an oxysulphide-based molecule.

8. The nanocrystal of claim 7, wherein the fluoride based molecule is sodium yttrium tetrafluoride (NaYF₄), Trifluoride yttrium (YF₃), Trifluoride lanthanum (LaF₃), or Trifluoride gadolinium (GdF₃).

9. The nanocrystal of claim 7, wherein the oxysulphide based molecule is yttrium oxisulphide (Y₂O₂5), lanthanum oxisulphide (La₂O₂5), or gadolinium oxisulphide (Gd₂O₂S).

10. The nanocrystal of claim 6, wherein the sensitizer is Ytterbium (Yb)

11. The nanocrystal of claim 6, wherein the emitter is Erbium (Er), Holmium (Ho), or Thulium (Tm).

12. The nanocrystal of claim 11, wherein the emitter concentration is between about 0.05 and 2.0 mol.

13. The nanocrystal of claim 1, wherein the nanocrystal size is between about 7.5 and 250 nm

14. The method of claim 2, wherein the excitation frequency is in the near infrared range.

15. A functionalized rare-earth ion doped, upconverting nanocrystal comprising:
a. a host molecule;
b. a rare earth ion sensitizer;
c. a rare earth ion emitter;
d. a coordination ligand capping the rare-earth ion doped nanocrystal, wherein the coordination ligand forces a substantially pure phase on the nanocrystal; and
e. a functionalizing coating, wherein the functionalizing coating does not affect the optical properties of the nanocrystal.

16. The functionalized nanocrystal of claim 15, whereupon excitation with an electromagnetic source, the functionalized nanocrystal emits an optical energy having a frequency that exceeds the excitation frequency.

17. The functionalized nanocrystal of claim 15, wherein the rare-earth ion sensitizer or emitter is a lanthanide ion combination.

18. The functionalized nanocrystal of claim 15, wherein the coordination ligand is:
an oleic acid/trioctylphosphine (OA/TOP) combination, or trioctylphosphine (TOPO).

19. The functionalized nanocrystal of claim 15, wherein the substantially pure phase of the functionalized nanocrystal is β-phase.

20. The functionalized nanocrystal of claim 17, wherein the lanthanide is Lanthanum (La), Cerium (Ce), Praseodymium (Pr), Neodymium (Nd) Promethium (Pm), Samarium (Sm), Europium (Eu), Gadolinium (Gd), Terbium (Tb) Dysprosium (Dy), Holmium (Ho), Erbium (Er), Thulium (Tm), Ytterbium (Yb), or Lutetium (Lu).

21. The functionalized nanocrystal of claim 15, wherein the host molecule is a fluoride-based or an oxysulphide-based molecule.

22. The functionalized nanocrystal of claim 21, wherein the fluoride based molecule is sodium yttrium tetrafluoride (NaYF₄), Trifluoride yttrium (YF₃), Trifluoride lanthanum (LaF₃), or Trifluoride gadolinium (GdF₃).

23. The functionalized nanocrystal of claim 21, wherein the oxysulphide based molecule is yttrium oxisulphide (Y₂O₂S), lanthanum oxisulphide (La₂O₂S), or gadolinium oxisulphide (Gd₂O₂S).

24. The functionalized nanocrystal of claim 20, wherein the sensitizer is Ytterbium (Yb)

25. The functionalized nanocrystal of claim 20, wherein the emitter is Erbium (Er), Holmium (Ho), or Thulium (Tm).

26. The functionalized nanocrystal of claim 25, wherein the emitter concentration is between about 0.05 and 2.0 mol.

27. The functionalized nanocrystal of claim 15, wherein the nanocrystal size is between about 7.5 and 250 nm.

28. The functionalized nanocrystal of claim 15, wherein the functionalizing coating is a silicate or an amphiphilic polymer.

29. The functionalized nanocrystal of claim 15, wherein the silicate is SiO₂.

30. The functionalized nanocrystal of claim 15, wherein the amphiphilic polymer is polyethylene glycol (PEG) or polyacrylic acid.

31. The functionalized nanocrystal of claim 15, further comprising a reagent operably linked to the functionalized nanocrystal

32. The functionalized nanocrystal of claim 31, wherein the reagent is an antibody or a functional fragment thereof, a small molecule, a toxin, a radioisotope, or their combination.

33. The functionalized nanocrystal of claim 32, wherein the antibody is a polyclonal antibody, a monoclonal antibody, a monospecific antibody, an alloantibody or a combination thereof.

34. The functionalized nanocrystal of claim 32, wherein the antibody fragment is a single-chain variable fragment (S_{c}fᵥ), F(ab), F′(ab), or F′(ab)₂.

35. The functionalized nanocrystal of claim 33, wherein the monospecific antibody or functional fragment thereof is specific against a red blood cells antigen.

36. The functionalized nanocrystal of claim 35, wherein the red blood cells antigen is an ABO family antigen, or an Rh family antigen.

37. A composition comprising the functionalized nanocrystal of claim 36.

38. A kit comprising: the composition of claim 37; an electromagnetic radiation source; an optical emission detector; a reagent; and instructions.

39. The kit of claim 37, wherein the electromagnetic radiation source emits radiation at the near infrared range.

40. A method of phenotyping red blood cells, comprising the steps of:
a. obtaining a blood sample;
b. contacting the blood sample with a composition comprising a first functionalized nanocrystal operably linked to an antibody or a functional fragment thereof, wherein the functionalized nanocrystal comprises:
i. a host molecule;
ii. a rare earth ion sensitizer;
iii. a rare earth ion emitter;
iv. a coordination ligand capping the rare-earth ion doped nanocrystal, wherein the coordination ligand forces a substantially pure phase on the nanocrystal; and
v. a functionalizing coating, wherein the functionalizing coating does not affect the optical properties of the nanocrystal;
c. exposing the blood sample contacted with the functionalized nanocrystal operably linked to an antibody or a functional fragment thereof to an electromagnetic radiation source; and
d. detecting an optical emission frequency, wherein the antibody or fragment thereof is specific against a red blood cells antigen, thereby phenotyping red blood cells.

41. The method of claim 40, whereby the composition further comprises an additional functionalized nanocrystal operably linked to an antibody or a functional fragment thereof, wherein the functionalized nanocrystal, wherein emission frequency of the additional functionalized is different than the first functionalized nanocrystal.

42. The method of claim 40, whereupon exposing the blood sample to an electromagnetic radiation source, the functionalized nanocrystal emits an optical energy having a frequency that exceeds the excitation frequency.

43. The method of claim 40, whereby the rare-earth ion sensitizer or emitter is a lanthanide ion combination.

44. The method of claim 40, whereby the coordination ligand is: an oleic acid/trioctylphosphine (OA/TOP) combination, or trioctylphosphine (TOPO).

45. The method of claim 40, whereby the substantially pure phase of the functionalized nanocrystal is a β-phase.

46. The method of claim 43, whereby the lanthanide is Lanthanum (La), Cerium (Ce), Praseodymium (Pr), Neodymium (Nd) Promethium (Pm), Samarium (Sm), Europium (Eu), Gadolinium (Gd), Terbium (Tb) Dysprosium (Dy), Holmium (Ho), Erbium (Er), Thulium (Tm), Ytterbium (Yb), or Lutetium (Lu).

47. The method of claim 40, whereby the host molecule is a fluoride-based or an oxysulphide-based molecule.

48. The method of claim 47, whereby the fluoride based molecule is sodium yttrium tetrafluoride (NaYF₄), Trifluoride yttrium (YF₃), Trifluoride lanthanum (LaF₃), or Trifluoride gadolinium (GdF₃).

49. The method of claim 47, whereby the oxysulphide based molecule is yttrium oxisulphide (Y₂O₂5), lanthanum oxisulphide (La₂O₂5), or gadolinium oxisulphide (Gd₂O₂S).

50. The method of claim 46, whereby the sensitizer is Ytterbium (Yb)

51. The method of claim 46, whereby the emitter is Erbium (Er), Holmium (Ho), or Thulium (Tm).

52. The method of claim 51, whereby the emitter concentration is between about 0.05 and 2.0 mol.

53. The method of claim 40, whereby the nanocrystal size is between about 7.5 and 250 nm

54. The method of claim 40, whereby the functionalizing coating is a silicate or an amphiphilic polymer.

55. The method of claim 40, whereby the silicate is SiO₂.

56. The method of claim 40, whereby the amphiphilic polymer is polyethylene glycol (PEG) or polyacrylic acid.

57. The method of claim 40, further comprising an antibody or a functional fragment thereof, operably linked to the functionalized nanocrystal.

58. The method of claim 40, whereby the antibody is a polyclonal antibody, a monoclonal antibody, a monospecific antibody, an alloantibody or a combination thereof.

59. The method of claim 40, whereby the antibody fragment is a single-chain variable fragment (S_{C}f_{V}), F(ab), F′(ab), or F′(ab)₂.

60. The method of claim 40, whereby the red blood cells antigen is an ABO family antigen, or an Rh family antigen.

61. The method of claim 58, whereby the alloantibody operably linked to the functionalized nanocrystal, is taken from a subject sought to be matched with the red blood cells phenotyped.

62. The method of claim 40, wherein the electromagnetic radiation source emits radiation at the near infrared range.

63. A method of matching donated red blood cells to a recipient comprising the steps of:
a. Obtaining a sample from the donated blood;
b. isolating an alloantiby panel from the recipient;
c. operably linking an isolated alloantibody from the panel to a first functionalized nanocrystal, wherein the functionalized nanocrystal comprises:
i. a host molecule;
ii. a rare earth ion sensitizer;
iii. a rare earth ion emitter;
iv. a coordination ligand capping the rare-earth ion doped nanocrystal, wherein the coordination ligand forces a substantially pure phase on the nanocrystal; and
v. a functionalizing coating, wherein the functionalizing coating does not affect the optical properties of the nanocrystal;
d. contacting the donated sample with the functionalized nanocrystal operably linked to the recepient's alloantibody;
e. exposing the blood sample contacted with the functionalized nanocrystal operably linked to the recepient' s alloantibody, to an electromagnetic radiation source; and
f. detecting an optical emission frequency, wherein the lower the intensity of the optical emission, the higher the match between the donated red blood cells and the recipient's

64. The method of claim 63, further comprising the step of contacting the donated sample with an additional functionalized nanocrystal operably linked to the recepient's alloantibody, wherein the emission frequency of the additional functionalized nanocrystal is different than the first functionalized nanocrystal.

65. The method of claim 64, whereby the emission frequency of the additional functionalized nanocrystal is the same as the first functionalized nanocrystal.

66. The method of claim 63, whereupon exposing the blood sample to an electromagnetic radiation source, the functionalized nanocrystal emits an optical energy having a frequency that exceeds the excitation frequency.

67. The method of claim 63, whereby the rare-earth ion sensitizer or emitter is a lanthanide ion combination.

68. The method of claim 63, whereby the coordination ligand is: an oleic acid/trioctylphosphine (OA/TOP) combination, or trioctylphosphine (TOPO).

69. The method of claim 63, whereby the substantially pure phase of the functionalized nanocrystal is a β-phase.

70. The method of claim 67, whereby the lanthanide is Lanthanum (La), Cerium (Ce), Praseodymium (Pr), Neodymium (Nd) Promethium (Pm), Samarium (Sm), Europium (Eu), Gadolinium (Gd), Terbium (Tb) Dysprosium (Dy), Holmium (Ho), Erbium (Er), Thulium (Tm), Ytterbium (Yb), or Lutetium (Lu).

71. The method of claim 63, whereby the host molecule is a fluoride-based or an oxysulphide-based molecule.

72. The method of claim 71, whereby the fluoride based molecule is sodium yttrium tetrafluoride (NaYF₄), Trifluoride yttrium (YF₃), Trifluoride lanthanum (LaF₃), or Trifluoride gadolinium (GdF₃).

73. The method of claim 71, whereby the oxysulphide based molecule is yttrium oxisulphide (Y₂O₂S), lanthanum oxisulphide (La₂O₂S), or gadolinium oxisulphide (Gd₂O₂S).

74. The method of claim 70, whereby the sensitizer is Ytterbium (Yb)

75. The method of claim 70, whereby the emitter is Erbium (Er), Holmium (Ho), or Thulium (Tm).

76. The method of claim 75, whereby the emitter concentration is between about 0.05 and 2.0 mol.

77. The method of claim 63, whereby the nanocrystal size is between about 7.5 and 250 nm

78. The method of claim 63, whereby the functionalizing coating is a silicate or an amphiphilic polymer.

79. The method of claim 63, whereby the silicate is SiO₂.

80. The method of claim 63, whereby the amphiphilic polymer is polyethylene glycol (PEG) or polyacrylic acid.

81. The method of claim 63, wherein the electromagnetic radiation source emits radiation at the near infrared range.

82. A method of performing direct antiglobulin test (DAT) on a subject comprising the steps of:
a. isolating erythrocytes from the subject;
b. isolating an immunoglobulin G (IgG) antibody panel from the subject;
c. operably linking the isolated immunoglobulin G (IgG) antibody from the panel to a first functionalized nanocrystal, wherein the functionalized nanocrystal comprises:
i. a host molecule;
ii. a rare earth ion sensitizer;
iii. a rare earth ion emitter;
iv. a coordination ligand capping the rare-earth ion doped nanocrystal, wherein the coordination ligand forces a substantially pure phase on the nanocrystal; and
v. a functionalizing coating, wherein the functionalizing coating does not affect the optical properties of the nanocrystal;
d. contacting the erythrocytes with the functionalized nanocrystal operably linked to the subject's immunoglobulin G (IgG) antibody;
e. exposing the blood sample contacted with the functionalized nanocrystal operably linked to the subject's immunoglobulin G (IgG) antibody, to an electromagnetic radiation source; and
f. detecting an optical emission frequency,
wherein the emission spectra indicates immunoglobulin attachment to the erythrocyte.

83. The method of claim 82, further comprising the step of contacting the isolated erythrocytes with an additional functionalized nanocrystal operably linked to the subject's isolated immunoglobulin G antibody, wherein the emission frequency of the additional functionalized nanocrystal is different than the first functionalized nanocrystal.

84. The method of claim 82, whereupon exposing the blood sample to an electromagnetic radiation source, the functionalized nanocrystal emits an optical energy having a frequency that exceeds the excitation frequency.

85. The method of claim 82, whereby the rare-earth ion sensitizer or emitter is a lanthanide ion combination.

86. The method of claim 82, whereby the coordination ligand is: an oleic acid/trioctylphosphine (OA/TOP) combination, or trioctylphosphine (TOPO).

87. The method of claim 82, whereby the substantially pure phase of the functionalized nanocrystal is a β-phase.

88. The method of claim 85, whereby the lanthanide is Lanthanum (La), Cerium (Ce), Praseodymium (Pr), Neodymium (Nd) Promethium (Pm), Samarium (Sm), Europium (Eu), Gadolinium (Gd), Terbium (Tb) Dysprosium (Dy), Holmium (Ho), Erbium (Er), Thulium (Tm), Ytterbium (Yb), or Lutetium (Lu).

89. The method of claim 82, whereby the host molecule is a fluoride-based or an oxysulphide-based molecule.

90. The method of claim 89, whereby the fluoride based molecule is sodium yttrium tetrafluoride (NaYF₄), Trifluoride yttrium (YF₃), Trifluoride lanthanum (LaF₃), or Trifluoride gadolinium (GdF₃).

91. The method of claim 89, whereby the oxysulphide based molecule is yttrium oxisulphide (Y₂O₂S), lanthanum oxisulphide (La₂O₂S), or gadolinium oxisulphide (Gd₂O₂S).

92. The method of claim 88, whereby the sensitizer is Ytterbium (Yb)

93. The method of claim 88, whereby the emitter is Erbium (Er), Holmium (Ho), or Thulium (Tm).

94. The method of claim 93, whereby the emitter concentration is between about 0.05 and 2.0 mol.

95. The method of claim 82, whereby the nanocrystal size is between about 7.5 and 250 nm

96. The method of claim 82, whereby the functionalizing coating is a silicate or an amphiphilic polymer.

97. The method of claim 82, whereby the silicate is SiO₂.

98. The method of claim 82, whereby the amphiphilic polymer is polyethylene glycol (PEG) or polyacrylic acid.

99. A method of functionalizing a rare-earth ion doped, upconverting nanocrystal, comprising the steps of coating a rare-earth ion doped, upconverting nanocrystal comprising:
a. a host molecule;
b. a rare earth ion sensitizer;
c. a rare earth ion emitter;
d. a coordination ligand capping the rare-earth ion doped nanocrystal,
wherein the coordination ligand forces a substantially pure phase on the nanocrystal,
with a functionalizing coating, wherein the functionalizing coating does not affect the optical properties of the nanocrystal.

100. The method of claim 99, whereupon excitation with an electromagnetic source, the functionalized nanocrystal emits an optical energy having a frequency that exceeds the excitation frequency.

101. The method of claim 99, wherein the rare-earth ion sensitizer or emitter is a lanthanide ion combination.

102. The method of claim 99, wherein the coordination ligand is: an oleic acid/trioctylphosphine (OA/TOP) combination, or trioctylphosphine (TOPO).

103. The method of claim 99, wherein the substantially pure phase of the functionalized nanocrystal is a β-phase.

104. The method of claim 101, wherein the lanthanide is Lanthanum (La), Cerium (Ce), Praseodymium (Pr), Neodymium (Nd) Promethium (Pm), Samarium (Sm), Europium (Eu), Gadolinium (Gd), Terbium (Tb) Dysprosium (Dy), Holmium (Ho), Erbium (Er), Thulium (Tm), Ytterbium (Yb), or Lutetium (Lu).

105. The method of claim 99, wherein the host molecule is a fluoride-based or an oxysulphide-based molecule.

106. The method of claim 105, wherein the fluoride based molecule is sodium yttrium tetrafluoride (NaYF₄), Trifluoride yttrium (YF₃), Trifluoride lanthanum (LaF₃), or Trifluoride gadolinium (GdF₃).

107. The method of claim 105, wherein the oxysulphide based molecule is yttrium oxisulphide (Y₂O₂5), lanthanum oxisulphide (La₂O₂S), or gadolinium oxisulphide (Gd₂O₂S).

108. The method of claim 104, wherein the sensitizer is Ytterbium (Yb)

109. The method of claim 104, wherein the emitter is Erbium (Er), Holmium (Ho), or Thulium (Tm).

110. The method of claim 109, wherein the emitter concentration is between about 0.05 and 2.0 mol.

111. The method of claim 99, wherein the nanocrystal size is between about 7.5 and 250 nm

112. The method of claim 99, wherein the functionalizing coating is a silicate or an amphiphilic polymer.

113. The method of claim 99, wherein the silicate is SiO₂.

114. The method of claim 99, wherein the amphiphilic polymer is polyethylene glycol (PEG) or polyacrylic acid

115. The method of claim 100, wherein the electromagnetic radiation source emits radiation at the near infrared range.
